SEQUENCE LISTING (2 sequences)

SEQ ID NO: 1 - protein, 1050 aa, Paenibacillus sp.
IAGVVQSVNVSQAGYSSNDFKTATVTASDKLSDTSYQILQGTTVIATGTMKDEGYVWGKYVYSIDFSSVTATGTNFTIRSNGVSSYTFPIQTNMWNEYKDEMTAFYRLLRTTDTFAAYPAGYSNIAPSNKILHPDSFLDDAFSPDRTTHYDLTGGWFDAGDYGKYGGNQWVQGNIAISYLRHASSAAVNFDKDTNGIPDLVDEAIFGSQYLVKFANQLGGAIHNILRKGGFVLPHKVTDNVPGNTDDRALEAVEAVGGSGKSSGSLAATARAIRTAIAGGKVAANKVAQLQTLANEFQAAAIIFYNYTLTHQSGNHGSYGTMNNGGIANPLLWAEVQLYLLTGDAAYKTQAQTRINAINEAYVSSTNYWDMHPIALAEFYPVADSAIKTKIQSILKHQAYYFITLMDETPYGVLNQFGNFGVNEPHASYMADLLRYYELFNDPVALRAAKKALYWIVGNNPWNISWVSGVGSNFTDFLHTRLDEEAYSQTNTGVVLPGAMVSGPNIKDPNNKLSSSPWYEDKPIWADDTNQWRYNEYSVSIQTGLFYTIMGLSALGGNASTGGAEPVKLPITWPIIGDYVTGDVTVFAQPEGSLSNVSANGIVLSPSDGVYTTTVSTSADAPYTERKVQIKGTDDSGFTTYSNTHFTVAPALPDPSHPLLFDDFNQKGIWGSQKLDWVNWYNQNGGTASYTRTTVDTRTVGKFAHTPAATTSKAKFQPWKYNANLNGYRYLNFTMKNPGYPNTKIRIAANDGTKSVNLTSGEVAISSTWTTYQYDLNLHPTLNKSNVLIEVWLSNPTAGAYGEILIDEISAVNTNSGTAPTLSATGVNASIGNQSTVFTYTATYTDANNQAPFDVQVVIDGVIRSMTAADPTDTTYSDGRVYTYATTLPVGTHKFYFRTTDTTTNFVSTSVQTGPTVIRNKLEAEVLSINLTNYTHAVKDNADASGGKYRLFNGRQANDYIEYAVNVPKAGTYQVSARAMRLSDNGIYQLQINGSNQGTPFDTYQSSGKYLDYALGNVTITSPGTQLFRFKVTGKNASSLGYKLPLDFIQ

SEQ ID NO: 2 - protein, 1037 aa, Paenibacillus sp.
DEFDTLREKYKAMLNGGTTYNLSDPDIAARVNAITVTAQGYWDSMLKDPNRNRLWNDAPFGSDSTSITTTYRHLYDMALAYTTYGSSLQGNAALKADIISGLDWMNANQFYNGCSQYQNWWHWQIGGPMALNDIVALMYTELTATQISNYMAAIYYTQASVTMTGANRLWESQVIAISGILNKDSARVAAGRDGISALLPYVAKGDGFYNDGSFVQHTYYAYNGGYGSELLSGIADLIFILNGSSWQVTDPNKNNVYRWIYDSYEPFIYKGNLMDMVRGREISRHGLQDDKAAVTVMASIIRLSQTAASADATAFKRMVKYWLLLDTDKTFLKAVSIDLIIAANQLVNDSTVTSRGELVKYKQFSGMDRAVQLRPGFGFGLSMFSSRIGNYESINAENNKGWHTGDGMTYLYNTDLSQFNDHFWATVDNYRLPGTTVLQNTTQTANSRSDKSWAGGTDILGQYGVSGMELHTVGKSLTAKKSWFMFDDEIVALGSGIASTDGIATETIVENRKLNSSGNNALIVNGTAKPGSLGWSETMTGTNYIHLAGSVPGSDIGYYFPGGAAVKGLREARSGSWSSLNSSASWKDSTLHTRNFMTLWFDHGMNPTNGSYSYVLLPNKTSSAVASYAATPQISILENSSSAQAVKETQLNVTGINFWNDEPTTVGLVTSNRKASVMTKETASDFEISVSDPTQSNVGTIYIDVNKSATGLISKDNEITVIQYYPTMKFKVNVNNSGGKSYKVKFSLTGTPGSNPSPIPIPNPYEAEALPINALTDTPVVYNDANASGGKKLGFNNNAVDDYVEFSLDVTQPGTYDVKSRIMKSTNSGIYQLSINGTNVGSAQDMFWTTSELSKEFTMGSYSFSTPGSYLFRLKTTGKNVSSSGYKLMLDNFSLVSTGIDTTVIVDNADAAGVTKVGTWTGTNTQTDRYGADYIHDGNTGKGTKSVTFTPNVPISGTYQVYMMWAAHTNRATNVPVDVTHSGGTATLNVNQQGNGGVWNLLGTYSFNAGSTGAIKIRTDATNGYVVADAVKLVKVP

(19) United States
(12) Patent Application Publication
(10) Pub. No.: US 2024/0093127 A1
(43) Pub. Date: Mar. 21, 2024

(54) Detergent composition comprising xanthan lyase and endoglucanase variants with improved stability

(71) Applicants: HENKEL AG & CO. KGAA, Duesseldorf (DE); NOVOZYMES A/S, Bagsvaerd (DK)

(72) Inventors: Christian DEGERING, Erkrath (DE); Inga Kerstin VOCKENROTH, Duesseldorf (DE); Brian LAUFS, Juechen (DE); Piotr TKOCZ, Monheim am Rhein (DE); Asbjørn Toftgaard PEDERSEN, Bagsvaerd (DK)

(21) Appl. No.: 18/272,104

(22) PCT Filed: Jan. 25, 2022
(86) PCT No.: PCT/EP2022/051528

§ 371 Date: Jul. 13, 2023

(30) Foreign Application Priority Data
Feb. 4, 2021 (EP) 21155245.0

(51) Int. Cl. C11D 3/386; C11D 3/20; C11D 11/00; C12N 9/42; C12N 9/88

(52) U.S. Cl. CPC C11D 3/38663; C11D 3/2079; C11D 3/38636; C11D 11/0017; C11D 11/0023; C12N 9/2437; C12N 9/88; C12Y 302/01004; C12Y 402/02012

(57) ABSTRACT

A detergent composition may include xanthan lyase and endoglucanase variants with improved stability. Methods for use of said compositions are also disclosed.

### CROSS-REFERENCE TO RELATED APPLICATIONS

The present application is a national stage entry according to 35 U.S.C. § 371 of PCT application No.: PCT/EP2022/051528 filed on Jan. 25, 2022; which claims priority to European patent application 21155245.0 filed on Feb. 4, 2021; all of which are incorporated herein by reference in their entirety and for all purposes.

### REFERENCE TO A SEQUENCE LISTING SUBMITTED VIA EFS-WEB

The content of the ASCII text file of the sequence listing named “P88036US_seq_ST25”, which is 18,240 bytes in size, was created on Feb. 4, 2021; the sequence listing is electronically submitted via EFS-Web herewith and is herein incorporated by reference in its entirety.

### JOINT RESEARCH AGREEMENT

The presently claimed invention was made by or on behalf of the below listed parties to a joint research agreement. The joint research agreement was in effect on or before the date the claimed invention was made, and the claimed invention was part of the joint research agreement and made as a result of activities undertaken within the scope of the joint research agreement. The parties to the joint research agreement are NOVOZYMES AS and HENKEL AG and Co KGaA.

### TECHNICAL FIELD

The present disclosure relates to a detergent composition, such as laundry compositions and dish wash compositions, including hand wash and automatic dish wash compositions, comprising xanthan lyase or endoglucanase or mixtures thereof and at least one enzyme stabilizing component, in order to improve at least one or more properties including detergent stability (e.g. improved stability in a detergent composition) and/or storage stability (e.g. improved storage stability in a detergent composition). The disclosure also relates to methods for producing and using such compositions as well as to the use of specific enzyme stabilizing components for improving storage stability of xanthan lyase or endoglucanase or mixtures thereof. The variants described herein are particularly suitable for use in cleaning processes and detergent compositions, in particular laundry detergents.

### BACKGROUND

Xanthan gum is a polysaccharide derived from the bacterial coat of Xanthomonas campestris. It is produced by the fermentation of glucose, sucrose, or lactose by the Xanthomonas campestris bacterium. After a fermentation period, the polysaccharide is precipitated from a growth medium with isopropyl alcohol, dried, and ground into a fine powder. Later, it is added to a liquid medium to form the gum. Xanthan gum is a natural polysaccharide consisting of different sugars which are connected by several different bonds, such as β-D-mannosyl-β-D-1,4-glucuronosyl bonds and β-D-glucosyl-β-D-1,4-glucosyl bonds. Xanthan gum is at least partly soluble in water and forms highly viscous solutions or gels. Complete enzymatic degradation of xanthan gum requires several enzymatic activities including xanthan lyase activity and endo-β-1,4-glucanase activity. Xanthan lyases are enzymes that cleave the β-D-mannosyl-β-D-1,4-glucuronosyl bond of xanthan and have been described in the literature. Xanthan lyases are known in the art, e.g. two xanthan lyases have been isolated from Paenibacillus alginolyticus XL-1 (e.g. Ruijssenaars et al. (1999) ‘A pyruvated mannose-specific xanthan lyase involved in xanthan degradation by Paenibacillus alginolyticus XL-1’, Appl. Environ. Microbiol. 65(6): 2446-2452, and Ruijssenaars et al. (2000), ‘A novel gene encoding xanthan lyase of Paenibacillus alginolyticus strain XL-1’, Appl. Environ. Microbiol. 66(9): 3945-3950).

Glycoside hydrolases are enzymes that catalyze the hydrolysis of the glycosyl bond to release smaller sugars. There are over 100 classes of glycoside hydrolases which have been classified, see e.g. Henrissat et al. (1991) ‘A classification of glycosyl hydrolases based on amino-acid sequence similarities’, J. Biochem. 280: 309-316 and the Uniprot website at www.cazy.org. The glycoside hydrolase family 9 (GH9) consists of over 70 different enzymes that are mostly endoglucanases (EC 3.2.1.4), cellobiohydrolases (EC 3.2.1.91), β-glucosidases (EC 3.2.1.21) and exo-β-glucosaminidase (EC 3.2.1.165).

In recent years, xanthan gum has been used as an ingredient in many consumer products including foods (e.g. as thickening agent in salad dressings and dairy products) and cosmetics (e.g. as stabilizer and thickener in toothpaste and make-up, creams and lotions to prevent ingredients from separating and to provide the right texture of the product). Further xanthan gum has found use in the oil industry as an additive to regulate the viscosity of drilling fluids etc. The widespread use of xanthan gum has led to a desire to degrade solutions, gels or mixtures containing xanthan gum thereby allowing easier removal of the byproducts. Endoglucanases and xanthan lyases for the degradation of xanthan gum and the use of such enzymes for cleaning purposes, such as the removal of xanthan gum containing stains, and in the drilling and oil industries are known in the art, e.g. WO 2013/167581, WO 2018/037064, WO 2018/037065 or WO 2019/162000.

The known endoglucanase having SEQ ID NO:1 and the known xanthan lyase having SEQ ID NO:2 were both found to be sensitive to the presence of detergents, in particular with respect to certain detergent components such as chelators and/or surfactants and/or further enzymes such as proteases. To improve the applicability and/or cost and/or performance of such enzymes, there is an on-going search for variants with altered properties, such as increased stability, e.g. improved stability in a detergent composition as such or improved stability after storage. However, mutagenesis of large enzymes followed by purification and functional analysis of mutant libraries can be very expensive and laborious. Hence, there is ongoing need for stabilized endoglucanase variants and/or xanthan lyase variants for the use in detergent compositions resp. for detergent compositions with improved enzyme stabilization, in particular for detergent compositions in which the enzyme mix containing endoglucanase and/or xanthan lyase shows improved stabilization.

### SUMMARY

In some aspects, the present disclosure relates to a detergent composition comprising

(A) an endoglucanase variant, comprising an alteration, preferably a substitution, at one or more positions in one or more regions selected from the group consisting of region 7 corresponding to amino acids 612 to 660 of SEQ ID NO:1, region 1 corresponding to amino acids 95 to 105 of SEQ ID NO:1, region 2 corresponding to amino acids 115 to 138 of SEQ ID NO:1, region 3 corresponding to amino acids 210 to 251 of SEQ ID NO:1, region 4 corresponding to amino acids 267 to 301 of SEQ ID NO:1, region 5 corresponding to amino acids 339 to 361 of SEQ ID NO:1, region 6 corresponding to amino acids 547 to 595 of SEQ ID NO:1, region 8 corresponding to amino acids 806 to 828 of SEQ ID NO:1, region 9 corresponding to amino acids 839 to 1042 of SEQ ID NO:1, region 10 corresponding to amino acids 1 to 94 of SEQ ID NO:1, region 11 corresponding to amino acids 106 to 114 of SEQ ID NO:1, region 12 corresponding to amino acids 139 to 209 of SEQ ID NO:1, region 13 corresponding to amino acids 252 to 266 of SEQ ID NO:1, region 14 corresponding to amino acids 302 to 338 of SEQ ID NO:1, region 15 corresponding to amino acids 362 to 546 of SEQ ID NO:1, region 16 corresponding to amino acids 596 to 611 of SEQ ID NO:1, region 17 corresponding to amino acids 661 to 805 of SEQ ID NO:1, region 18 corresponding to amino acids 829 to 838 of SEQ ID NO:1, and region 19 corresponding to amino acids 1043 to 1055 of SEQ ID NO:1; and/or

(B) a xanthan lyase variant, comprising an alteration, preferably a substitution, at one or more positions in one or more regions selected from the group consisting of region 3 corresponding to amino acids 731 to 803 of SEQ ID NO:2, region 1 corresponding to amino acids 154 to 176 of SEQ ID NO:2, region 2 corresponding to amino acids 614 to 658 of SEQ ID NO:2, region 4 corresponding to amino acids 807 to 846 of SEQ ID NO:2, region 5 corresponding to amino acids 872 to 885 of SEQ ID NO:2, region 6 corresponding to amino acids 903 to 1004 of SEQ ID NO:2, region 7 corresponding to amino acids 1 to 153 of SEQ ID NO:2, region 8 corresponding to amino acids 177 to 613 of SEQ ID NO:2, region 9 corresponding to amino acids 659 to 730 of SEQ ID NO:2, region 10 corresponding to amino acids 804 to 806 of SEQ ID NO:2, region 11 corresponding to amino acids 847 to 871 of SEQ ID NO:2, region 12 corresponding to amino acids 886 to 902 of SEQ ID NO:2, and region 13 corresponding to amino acids 1005 to 1037 of SEQ ID NO:2; and

(C) an enzyme stabilizing component.

In various aspects, the endoglucanase variant (A) has at least 60% and less than 100% sequence identity to SEQ ID NO:1; preferably said endoglucanase variant has activity on xanthan gum pre-treated with xanthan lyase; and/or the xanthan lyase variant (B) has at least 60% and less than 100% sequence identity to SEQ ID NO:2, preferably said xanthan lyase variant having an activity on xanthan gum.

In further aspects, the enzyme stabilizing component is selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and sodium formate, more preferably being sodium formate.

In some aspects, the detergent composition, as defined herein, comprises an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and less than 100% sequence identity to SEQ ID NO:1.

In some aspects, the detergent composition, as defined herein, comprises a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and less than 100% sequence identity to SEQ ID NO:2.

In some aspects, the detergent composition comprises an endoglucanase variant comprising an alteration, preferably a substitution, at one or more positions selected from the group consisting of 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 719, 720, 744, 749, 754, 756, 760, 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045 and 1048, preferably at one or more positions selected from the group consisting of 17, 20, 216, 283, 302, 311, 313, 408, 476, 579, 602, 627, 636, 638, 651, 688, 697, 719, 756, 880, 881, 883, 887, 906, 921, 934, 937, and 956, wherein said positions correspond to amino acid positions of SEQ ID NO:1.

In some aspects, the detergent composition comprises a xanthan lyase variant comprising an alteration, preferably a substitution, at one or more positions selected from the group consisting of 9, 15, 18, 46, 58, 66, 89, 95, 100, 106, 109, 155, 159, 183, 188, 190, 203, 204, 221, 229, 234, 238, 240, 242, 243, 257, 258, 284, 291, 293, 316, 317, 320, 324, 329, 333, 339, 341, 352, 354, 360, 372, 377, 399, 400, 419, 440, 450, 451, 454, 458, 481, 492, 505, 533, 567, 568, 576, 578, 579, 582, 620, 624, 626, 631, 635, 649, 650, 656, 664, 672, 703, 722, 726, 727, 728, 738, 745, 752, 753, 754, 757, 769, 775, 777, 779, 782, 786, 789, 792, 796, 799, 801, 819, 824, 843, 851, 855, 856, 867, 875, 887, 892, 899, 900, 901, 902, 903, 911, 912, 915, 919, 921, 923, 925, 927, 928, 930, 933, 941, 966, 991, 1008 and 1016, wherein said positions correspond to amino acid positions of SEQ ID NO:2.

In some aspects, the aforementioned variants have at least 60% and less than 100% sequence identity to SEQ ID NO:2, preferably said xanthan lyase variant having an activity on xanthan gum.

In some aspects, the detergent composition comprises an enzyme stabilizing component being selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate.

In particular aspects, the detergent composition comprises an enzyme stabilizing component being selected from the group consisting of glycerol and sodium formate, more preferably sodium formate.

In some aspects, the detergent composition comprises an endoglucanase variant comprising an alteration, preferably a substitution, at one or more positions in: (i) regions 6 and 17; (ii) regions 6, 15 and 17; (iii) regions 10, 12 and 15; (iv) regions 6, 7, 16, and 17; (v) region 6, 9, 10, 12, 15, and 17; (vi) region 14 and 15; (vii) region 9; (viii) 6, 7, 9, 14, 15, 16, and 17; or (ix) 3, 6, 7, 9, 14, 15, 16, and 17; wherein said variant preferably has no alternation in the other regions besides those mentioned.

In further aspects, the detergent composition comprises an endoglucanase variant comprising one or more amino acid substitutions selected from the group consisting of V4T, S17A, N18G, F20P, F20N, F20G, F20Y, K51Q, K51H, E53Y, E53P, E53G, Y55M, Y55D, V56M, Y60F, S63F, A71E, T87R, T92S, K99R, A120P, I125V, A126R, S128X, N129D, K130R, F137L, H182Y, A186P, N189K, K192N, K213R, N216D, N216Q, N216R, A221R, L226K, K228E, K228I, G230F, G230L, G230A, G230H, G230N, G230W, G230T, F231Y, F231N, V232R, V232G, L233H, H235D, N240Q, G243K, G243R, D247N, A249N, A278S, G279E, K281F, K281V, K281Y, K281H, K281Q, K281N, K281W, K281T, K281R, A283D, N285G, N285L, N285M, N285S, N285P, N285T, N285Y, N285H, N285K, N285D, N285W, N285R, Q289E, T292A, T292F, T292L, T292I, T292V, T292S, T292P, T292Y, T292Q, T292N, T292K, T292D, T292G, A294V, F297L, Q298E, I302D, I302H, I302V, I302M, H311N, S313D, W333L, A346H, A346D, T353D, A386P, I387T, K388R, K390Q, I403Y, E408D, E408N, E408S, E408P, E408A, E408G, P410G, Q416S, Q416D, N441G, A448E, A448W, A448S, K451Q, K451S, G471S, S472Y, D476R, Q489P, K507R, K512P, S515V, S538C, L555Q, G556S, G557R, N558P, N558F, N558K, N558D, N558M, N558Q, N558I, N558Y, N558H, N558E, A559N, A559F, A559M, A559P, A559Y, A559H, A559Q, A559D, A559R, A559G, A559I, A559S, A559K, S560F, S560P, S560K, S560G, S560D, T561E, T561Q, T561S, T561D, T561P, G562W, G562P, G563E, A564I, A564Y, A564H, A564Q, A564K, A564E, A564D, E565M, V567F, V567P, K568R, L569F, L569Y, L569D, L569E, P570F, P570L, P570I, P570M, P570V, P570S, P570T, P570A, P570Y, P570H, P570Q, P570N, P570K, P570E, P570W, P570R, P570G, I575V, I575M, I575A, I575D, I575E, I576F, I576M, I576P, D578R, Y579F, Y579W, V580L, T581M, D583M, Q589G, P590S, P590T, P590E, E591L, G592D, S593P, S593H, S593Q, S593N, S593K, S593D, S593E, S593R, S595L, S598Q, A599S, I602T, I602D, V603P, S605T, S607C, G609E, S616G, S616D, K627L, K627M, K627V, K627S, K627T, K627Q, K627R, I630F, I630V, I630Y, K631R, K631A, T633V, D635L, D635M, D635T, D635A, D635P, D635N, D635K, D635E, D635G, D635W, S636L, S636M, S636A, S636H, S636Q, S636N, S636K, S636R, F638N, F638Y, T639D, F638I, F638V, F638T, F638L, F638H, F638M, T639V, T639S, T639L, T639I, T639M, T639A, T639E, T639W, T639G, T639Y, T639P, T640S, Y641E, S642T, S642N, N643D, N643H, N643T, T644F, A651P, A651S, D676H, Q683E, A688G, Y690F, T694A, T697G, R698W, T699A, T706Q, T711S, T711V, T711Y, K713R, W719R, K720H, K744H, K744Q, A749T, K754R, V756Y, V756H, S760G, T781M, N786K, T797S, S810Q, S810R, A811S, V812F, V812I, V812M, V812W, V812R, N815V, N815Y, N815E, N815W, N815R, S823Q, A824T, A824D, T825G, T825N, T825W, T825A, T825D, V827I, V827M, V827S, N828D, N833D, Q834E, S835A, S835D, V837I, T843V, N848D, A868E, A869V, D870F, D870L, D870I, D870M, D870V, D870S, D870T, D870Y, D870H, D870Q, D870N, D870K, D870E, D870W, D870R, D870G, P871F, P871L, P871I, P871M, P871V, P871S, P871T, P871A, P871Y, P871H, P871Q, T872S, T872F, T872A, T872Y, T872H, T872Q, T872N, T872K, T872D, T872E, T872W, T872R, T872G, D873K, D873E, T874V, T874S, T874P, T874A, T874H, T874Q, T874N, T874K, R880K, V881Q, V881T, T883R, T883V, T883C, T883K, Y884H, A885F, A885Q, A885N, T887L, T887I, T887S, T887H, T887R, T887K, L888M, V890R, T892P, T892V, K894E, R898Q, N905D, F906A, Q912V, N920P, N920D, K921R, K921E, K921D, A924D, V926F, V926P, K927R, S928D, T932A, N933V, N933S, Y934G, Y934M, Y934S, Y934A, Y934Q, Y934N, Y934E, Y934W, Y934R, T935W, A937F, A937V, A937S, A937T, A937Q, A937D, A937E, V938I, K939I, K939V, D940E, N941S, N941H, N941D, A942P, A942E, D943Y, D943H, G946R, K948R, K948E, R950V, R950H, R950N, F952S, F952W, N953Y, G954L, Q956Y, Q956S, A957L, A957P, Y960F, A964N, A964C, N966P, N966C, G971A, T972K, Q974K, Q974C, M980I, Q989I, Q991L, Q991I, Q991M, Q991V, Q991T, Q991K, Q991C, G994D, G994N, S995I, S995V, S995Q, S995R, S995C, G998V, G998A, T999R, S1006T, S1006A, S1006K, S1006R, K1009E, Y1010W, L1011M, L1011S, L1011A, L1011Q, L1011N, L1011D, L1011E, R1029N, F1030M, K1031I, K1031S, K1031T, K1031H, V1032G, K1035A, A1037E, A1037W, S1038L, S1038I, S1038G, L1040N, L1040E, G1041F, G1041R, Y1042N, L1044F, L1044S, L1044N, L1044W, P1045Q, P1045W, and F1048W, wherein said positions correspond to amino acid positions of SEQ ID NO:1.

In further aspects, the detergent composition comprises an endoglucanase variant comprising a set of alterations selected from the group consisting of A559K+N558K+S560F+T561P+G562W, A559N+P570Q, A559N+P570T, A559N+Y579F, A559N+Y579F+K627R, A559N+Y579W, A559N+Y579W+K99R, A559N+Y579W+S128X+K627R, A559N+Y579W+S128X+S636N, A559N+Y579W+K281R, A559N+Y579W+Q416D+S636N, A559N+Y579W+S560P, A559N 30 Y579W+A564I, A559N+Y579W+P570N, A559N+Y579W+P570K, A559N+Y579W+P570R, A559N+Y579W+P570A, A559N+Y579W+P570T, A559N+Y579W+P570S, A559N+Y579W+P570Q, A559N+Y579W+P570H, A559N+Y579W+S616D, A559N+Y579W+S616D+K627R, A559N+Y579W+S616D+S636K, A559N+Y579W+S616D+A651P, A559N+Y579W+S616D+K921R, A559N+Y579W+S616D+K921R+Y934G, A559N+Y579W+K627R, A559N+Y579W+K627R+S636N+A651P, A559N+Y579W+K627R+S636N+K921R, A559N+Y579W+K627R+K921R, A559N+Y579W+K627M, A559N+Y579W+K627M+A651P, A559N+Y579W+S636N, A559N+Y579W+S636N+A651P, A559N+Y579W+S636N+K921R, A559N+Y579W+S636K+A651P, A559N+Y579W+S636K+K921R, A559N+Y579W+S636L, A559N+Y579W+F638I, A559N+Y579W+A651P, A559N+Y579W+A651P+K921R+Y934G, A559N+Y579W+A651P+Y934G, A559N+Y579W+D870M, A559N+Y579W+K921R, A559N+Y579W+K921R+A651P, A559N+Y579W+K921R+Y934G, A559N+Y579W+Y934G, A559N+Y579W+K948E, A559N+Y579W+K1009E, A559N+K627R, Y579F+A564E, Y579W+K99R, Y579W+G562P, Y579W+A564D, 579W+P570K, Y579W+P570Q, Y579W+K627R, Y579W+S636N, Y579W+F638I, Y579W+A651P, Y579W+K921R, Y579W+Y934G, Y579W+K948E, Y579W+K1009E, K627R+K51Q, K627R+K451S, K627R+P570Q, K627R+P570T, K627R+S636N, K627R+F638I, K627R +A885F, K627R+Y934G, S636N+P570T, S636N+K921R, S636N+Y934G, F638I+P570K, F638I+P570Q, F638I+P570T, F638I+A651P, F638I+Y934G, F638I+K921R, A651P+P570Q, A651P+P570T, A885F+P570T, A885F+Y934G, K921R+P570Q, K921R+P570T, Y934G+P570T, wherein said positions correspond to amino acid positions of SEQ ID NO:1.

In a preferred aspect, the detergent composition comprises an endoglucanase variant comprising an alteration, preferably a substitution, at one or more positions selected from the group consisting of S17A, F20P, N216D, N216Q, A283D, I302D, H311N, S313D, E408D, D476R, Y579W, I602T, K627R, S636N, S636K F638I, F638N, A651P, A688G, T697G, W719R, V756Y, R880K, V881Q, T883R, T887K, F906A, K921R, Y934G, A937E, and Q956Y, wherein said positions correspond to amino acid positions of SEQ ID NO:1.

In a further preferred aspect, the detergent composition comprises an endoglucanase variant comprising a set of alterations selected from the group consisting of A559N+Y579W+T697G, K512P+A559N+Y579W+T697G, N18G+A71E+A186P+E408D+Y579W+I602T+A651P+A688G+V756Y, N18G+N189K+E408D+A559N+Y579W+A688G+T697G+V756Y+K921R+Y934G, S313D+E408D, R880K+N905D+K921R+Y934G, I302D+S313D+E408D+Y579W+I602T+A651P+T697G+R880K+K921R+Y934G, and N216Q+S313D+E408D+D476R+Y579W+I602T+F638N+A651P+T697G+W719R+R880K+T887K+K921R+Y934G, wherein said positions correspond to amino acid positions of SEQ ID NO:1.

In some aspects, the detergent composition comprises an endoglucanase variant having activity on xanthan gum pre-treated with xanthan lyase; preferably said activity comprises endoglucanase EC 3.2.1.4 activity, further preferably said activity is endoglucanase EC 3.2.1.4 activity.

In some aspects, the detergent composition comprises an isolated GH9 endoglucanase variant having activity on xanthan gum pre-treated with xanthan lyase.

In some aspects, the xanthan lyase variant, as described herein, is one that comprises an alteration, preferably a substitution, at one or more positions in: (i) regions 3 and 5; (ii) regions 3, 5 and 12; (iii) regions 8, and 9; (iv) regions 2, 3, and 5; (v) regions 2, 3, 5, and 12; (vi) regions 3, 5, 8, 9, and 12; (vii) regions 2, 3, 5, 8, and 9; (viii) 3, 5, 8, 9, and 12; (ix) 2, 3, 5, 8, 9, and 12; (x) region 3; (xi) regions 3, 4 and 5; (xii) regions 7, 8 and 9; (xiii) regions 12 and 13; (xiv) regions 3, 4, 5, 8, 9, and 12; (xv) regions 8, 9, 12, and 13; (xvi) regions 7, 8, 9, 12, and 13; (xvii) regions 3, 4, 5, 7, 8, 9, and 12; and (xviii) regions 3, 4, 5, 7, 8, 9, 12, and 13, wherein said variant preferably has no alteration in the other regions besides those mentioned.

In further aspects, the detergent composition comprises a xanthan lyase variant comprising one or more amino acid substitutions selected from the group consisting of K9R, N15T, T18D, L46D, A58L, S66H, Q89Y, K95E, S100D, N106Y, Q109R, Q109D, Q109F, Q109K, Q109A, Y155E, A159P, K183Q, K183R, V188I, A190Q, A203P, K204R, A221P, E229N, E229S, E229V, I234V, I238W, I238L, I238M, 1240W, N242S, G243V, Y257W, R258E, R284G, K291R, A293G, A293P, K316R, R317K, K320R, L324Q, K329R, K333R, L339M, I341P, V352I, S354P, K360G, K360R, Q372H, F377Y, N399K, K400R, F419Y, N440K, D450P, K451E, K451R, A454V, D458S, K481R, A492H, A492L, T505I, L533I, K567R, G568A, S578K, S578N, S578R, S579R, S579K, S582K, K620R, A624E, A626Q, T631N, S635E, S635T, T649V, T649K, Q650G, I656V, T664K, N672D, I703L, I722F, P726Q, T727P, M728V, G738L, K745R, P752R, P752K, G753E, G753Q, G753S, S754E, S754L, S754Q, S754R, S757D, S757P, S757E, A769D, A769T, A769R, L775A, L775F, L775I, L775M, L775Q, L775S, L775Y, D777R, P779V, Y782I, N786K, G789R, K792W, K792Y, K792V, K792A, N796Q, A799H, D801G, K819R, K824R, A843P, S851F, K855R, E856D, P867S, K875T, K875E, K887R, N892Y, N892W, N892F, G899S, I900G, D901A, T902F, T903A, T903Q, A911M, A911V, A911S, A912T, A912I, A912Y, T915S, T915V, T915Q, T919D, T919F, T919G, T921R, T921S, T923D, T923H, T925D, T925Q, T925R, T927K, D928W, Y930F, Y930H, Y930L, D933M, G941D, G941E, A966P, N991D, V998K, N1008D and K1016T, wherein said positions correspond to amino acid positions of SEQ ID NO:2.

In a preferred aspect, the detergent composition comprises a xanthan lyase variant comprising an alteration at one or more positions selected from the group consisting of 624, 631, 635, 649, 656, 738, 752, 753, 754, 757, 769, 775, 777, 800, 801, 843, 875, 911 and 915, and/or an alteration at one or more positions selected from the group consisting of 89, 100, 190, 229, 234, 352, 360, 399, 440, 458, 492, 567, 582, 664, 672, 703, 728, 892, 1008 and 1016, wherein said positions correspond to amino acid positions of SEQ ID NO:2.

In a further preferred aspect, the detergent composition comprises a xanthan lyase variant comprising one or more substitutions selected from the group consisting of Q89Y, S100D, A190Q, E229S, I234V, V352I, K360G, N399K, N440K, D458S, A492H, A492L, K567R, S582K, T664K, N672D, I703L, M728V, N892Y N1008D and K1016T, and/or one or more substitutions selected from the group consisting of A624E, T631N, S635E, T649K, 1656V, G738L, P752K, P752R, G753E, S754E, S754R, S757D, A769D, L775A, D777R, V800P, D801G, A843P, K875T, A911V and T915A, wherein said positions correspond to amino acid positions of SEQ ID NO:2.

Non-limiting examples of such variants include: A190Q+E229S+S635E+T649K+I656V+N672D+I703L+G753E+S754E+S757D+A769D+L775A+D801G+K875T+N892Y+N1008D, E229S+S635E+T649K+I656V+N672D+I703L+G753E+S754E+A769D+L775A+D801G+K875T+N892Y+N1008D, E229S+V352I+S635E+T649K+I656V+N672D+G753E+S754E+A769D+L775A+V800P+D801G+K875T+N892Y, E229S+K360G+D458S+S582K+N672D+G753E+S754E+A769D+L775A+D801G+K875T+N892Y+N1008D, S100D+E229S+K360G +D458S+S582K+T664K+N672D+G753E+S754E+S757D+A769D+L775A+D801G+A843P+K875T+N892Y+A911V+N1008D+K1016T, E229S+I234V+S582K+N672D+G753E +S754E+A769D+L775A+V800P+D801G+K875T+N892Y, Q89Y+E229S+N440K+S582K+A624E+N672D+G753E+S754E+A769D+L775A+D801G+K875T+N892Y, E229S+S635E+T649K+I656V+N672D+P752K+G753E+A769D+L775A+D801G+A843P+K875T+N892Y, E229S+S635E+T649K+I656V+N672D+G753E+S754E+S757D+A769D+L775A+D801G+K875T+N892Y, E229S+N440K+S582K+N672D+G753E+S754E+A769D+L775A+D801G+A843P+K875T+N892Y+N1008D, E229S+N440K+S582K+A624E+N672D+G753E+S754E +A769D+L775A+V800P+D801G+K875T+N892Y, A190Q+E229S+S635E+T649K+I656V+N672D+P752K+G753E+A769D+L775A+D801G+A843P+K875T+N892Y, A190Q+E229S+S582K+N672D+G753E+S754E+A769D+L775A+D801G+K875T+N892Y, E229S+N440K+S582K+N672D+P752R+G753E+S754E+S757D+A769D+L775A+D801G+K875T+N892Y +N1008D, E229S+S582K+S635E+N672D+P752R+G753E+S754E+A769D+L775A+D801G+K875T+N892Y+N1008D, A190Q+E229S+N440K+S582K+A624E+S635E+N672D +G753E+S754E+A769D+L775A+D801G+K875T+N892Y, E229S+I234V+A492L+S582K+N672D+G753E+S754E+A769D+L775A+D801G+K875T+N892Y, A190Q+E229S+K360G+D458S+S582K+T664K+N672D+G753E+S754E+A769D+L775A+D801G+K875T+N892Y +N1008D, S100D+E229S+K360G+D458S+S582K+N672D+G753E+S754E+S757D+A769D+L775A+D801G+A843P+K875T+N892Y+T915A+N1008D, E229S+N440K+S582K +A624E+S635E+N672D+G738L+G753E+S754E+S757D+A769D+L775A+D801G+K875T +N892Y, S100D+E229S+K360G+D458S+S582K+N672D+G753E+S754E+A769D+L775A +D801G+K875T+N892Y+N1008D, A190Q+E229S+D458S+T631N+N672D+G753E+S754E+A769D+L775A+D801G+A843P+K875T+N892Y, A190Q+E229S+K360G+D458S+S582K+N672D+G753E+S754E+A769D+L775A+D801G+K875T+N892Y+N1008D, E229S +S635E+T649K+I656V+N672D+G753E+S754R+S757D+A769D+L775A+D801G+A843P +K875T+N892Y, E229S+D458S+S582K+T631N+S635E+N672D+M728V+G753E+S754E +S757D+A769D+L775A+D801G+K875T+N892Y, A190Q+E229S+K360G+D458S+S582K +N672D+G753E+S754E+S757D+A769D+L775A+D801G+K875T+N892Y+N1008D, E229S+A492L+S635E+T649K+I656V+N672D+G753E+S757D+A769D+L775A+D801G+K875T+N892Y, S100D+A190Q+E229S+K360G+D458S+S582K+N672D+G753E+S754E+A769D+L775A+D801G+K875T+N892Y+N1008D, A190Q+E229S+I234V+S582K+N672D+G753E+S754E+S757D+A769D+L775A+D801G+K875T+N892Y, E229S+N399K+D458S+A492H+K567R+S582K+S635E+T649K+N672D+G753E+S754E+A769D+L775A+D777R +D801G+K875T+N892Y, E229S+D458S+A492L+T631N+N672D+G753E+S754E+S757D +A769D+L775A+D801G+K875T+N892Y, E229S+D458S+A492H+K567R+S582K+S635E +N672D+G753E+S754E+A769D+L775A+D777R+D801G+K875T+N892Y, S100D+E229S +K360G+D458S+S582K+N672D+G753E+S754E+S757D+A769D+L775A+D801G+

A843P+K875T+N892Y+N1008D, E229S+N399K+D458S+K567R+S582K+S635E+N672D +G753E+S754E+A769D+L775A+D777R+D801G+K875T+N892Y.

In a preferred aspect, the detergent composition comprises a xanthan lyase variant comprising a set of alterations selected from the group consisting of E229N+N672D+P752K+G753E+A769D +L775A+D801G+K875T+N892Y, E229S+N672D+G753E+S754E+A769D+L775A+D801G +K875T+N892Y, E229S+N672D+P752R+G753E+S754E+A769D+L775A+D801G+K875T +N892Y, A190Q+E229S+I234V+A624E+N672D+G753E+S754E+A769D+L775A+D801G +K875T, A190Q+E229S+T631N+N672D+I703L+P752K+G753E+A769D+L775A+D801G +K875T, A190Q+E229S+I234V+S582K+N672D+G753E+S754E+S757D+A769D+L775A +D801G+K875T+N892Y, E229S+N440K+S582K+A624E+S635E+N672D+G738L+G753E +S754E+S757D+A769D+L775A+D801G+K875T+N892Y, and S100D+E229S+K360G+D458S+S582K+N672D+G753E+S754E+S757D+A769D+L775A+D801G+A843P+K875T +N892Y+N1008D, wherein said positions correspond to amino acid positions of SEQ ID NO:2.

In some aspects, the detergent composition comprises a xanthan lyase variant having activity on xanthan gum; preferably said activity comprises xanthan lyase EC 4.2.2.12 activity, further preferably said activity is xanthan lyase EC 4.2.2.12 activity.

In some aspects, the detergent composition, as defined herein, comprising an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of A559K+N558K+S560F+T561P+G562W, A559N+P570Q, A559N+P570T, A559N+Y579F, A559N+Y579F+K627R, A559N+Y579W, A559N+Y579W+K99R, A559N+Y579W+S128X+K627R, A559N+Y579W+S128X+S636N, A559N+Y579W+K281R, A559N+Y579W+Q416D+S636N, A559N+Y579W+S560P, A559N+Y579W+A564I, A559N+Y579W+P570N, A559N+Y579W+P570K, A559N+Y579W+P570R, A559N+Y579W+P570A, A559N+Y579W+P570T, A559N+Y579W+P570S, A559N+Y579W+P570Q, A559N+Y579W+P570H, A559N+Y579W+S616D, A559N+Y579W+S616D+K627R, A559N+Y579W+S616D+S636K, A559N+Y579W+S616D+A651P, A559N+Y579W+S616D+K921R, A559N+Y579W+S616D+K921R+Y934G, A559N+Y579W+K627R, A559N+Y579W+K627R+S636N+A651P, A559N+Y579W+K627R+S636N+K921R, A559N+Y579W+K627R+K921R, A559N+Y579W+K627M, A559N+Y579W+K627M+A651P, A559N+Y579W+S636N, A559N+Y579W+S636N+A651P, A559N+Y579W+S636N+K921R, A559N+Y579W+S636K+A651P, A559N+Y579W+S636K+K921R, A559N+Y579W+S636L, A559N+Y579W+F638I, A559N+Y579W+A651P, A559N+Y579W+A651P+K921R+Y934G, A559N+Y579W+A651P+Y934G, A559N+Y579W+D870M, A559N+Y579W+K921R, A559N+Y579W+K921R+A651P, A559N+Y579W+K921R+Y934G, A559N+Y579W+Y934G, A559N+Y579W+K948E, A559N+Y579W+K1009E, A559N+K627R, Y579F+A564E, Y579W+K99R, Y579W+G562P, Y579W+A564D, 579W+P570K, Y579W+P570Q, Y579W+K627R, Y579W+S636N, Y579W+F638I, Y579W+A651P, Y579W+K921R, Y579W+Y934G, Y579W+K948E, Y579W+K1009E, K627R+K51Q, K627R+K451S, K627R+P570Q, K627R+P570T, K627R+S636N, K627R+F638I, K627R+A885F, K627R+Y934G, S636N+P570T, S636N+K921R, S636N+Y934G, F638I +P570K, F638I+P570Q, F638I+P570T, F638I+A651P, F638I+Y934G, F638I+K921R, A651P+P570Q, A651P+P570T, A885F+P570T, A885F+Y934G, K921R+P570Q, K921R+P570T, Y934G +P570T, preferably selected from the group consisting of A559N+Y579W+T697G, K512P+A559N +Y579W+T697G, N18G+A71E+A186P+E408D+Y579W+I602T+A651P+A688G+V756Y, N18G+N189K+E408D+A559N+Y579W+A688G+T697G+V756Y+K921R+Y934G, S313D+E408D, R880K+N905D+K921R+Y934G, I302D+S313D+E408D+Y579W+I602T+A651P+T697G+R880K+K921R+Y934G, and N216Q+S313D+E408D+D476R+Y579W+I602T+F638N+A651P+T697G+W719R+R880K+T887K+K921R+Y934G, wherein said positions correspond to amino acid positions of SEQ ID NO:1.

In a particular aspect, the endoglucanase variant has besides the aforementioned alterations no further alterations relative to the parent enzyme of SEQ ID NO:1, i.e. the remaining sequence is identical to SEQ ID NO:1.

In some aspects, the detergent composition, as defined herein, comprises a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of A190Q+E229S+S635E+T649K+I656V+N672D+I703L+G753E+S754E+S757D+A769D+L775A+D801G+K875T+N892Y+N1008D, E229S+S635E+T649K+I656V+N672D+I703L+G753E+S754E+A769D+L775A+D801G+K875T+N892Y+N1008D, E229S+V352I+S635E+T649K+I656V+N672D+G753E+S754E+A769D+L775A+V800P+D801G+K875T+N892Y, E229S+K360G+D458S+S582K+N672D+G753E+S754E+A769D+L775A+D801G+K875T+N892Y+N1008D, S100D+E229S+K360G +D458S+S582K+T664K+N672D+G753E+S754E+S757D+A769D+L775A+D801G+A843P+K875T+N892Y+A911V+N1008D+K1016T, E229S+I234V+S582K+N672D+G753E +S754E+A769D+L775A+V800P+D801G+K875T+N892Y, Q89Y+E229S+N440K+S582K+A624E+N672D+G753E+S754E+A769D+L775A+D801G+K875T+N892Y, E229S+S635E+T649K+I656V+N672D+P752K+G753E+A769D+L775A+D801G+A843P+K875T+N892Y, E229S+S635E+T649K+I656V+N672D+G753E+S754E+S757D+A769D+L775A+D801G+K875T+N892Y, E229S+N440K+S582K+N672D+G753E+S754E+A769D+L775A+D801G+A843P+K875T+N892Y+N1008D, E229S+N440K+S582K+A624E+N672D+G753E+S754E +A769D+L775A+V800P+D801G+K875T+N892Y, A190Q+E229S+S635E+T649K+I656V+N672D+P752K+G753E+A769D+L775A+D801G+A843P+K875T+N892Y, A190Q+E229S+S582K+N672D+G753E+S754E+A769D+L775A+D801G+K875T+N892Y, E229S+N440K+S582K+N672D+P752R+G753E+S754E+S757D+A769D+L775A+D801G+K875T+N892Y +N1008D, E229S+S582K+S635E+N672D+P752R+G753E+S754E+A769D+L775A+D801G+K875T+N892Y+N1008D, A190Q+E229S+N440K+S582K+A624E+S635E+N672D +G753E+S754E+A769D+L775A+D801G+K875T+N892Y, E229S+I234V+A492L+S582K+N672D+G753E+S754E+A769D+L775A+D801G+K875T+N892Y, A190Q+E229S+K360G+D458S+S582K+T664K+N672D+G753E+S754E+A769D+L775A+D801G+K875T+N892Y +N1008D, S100D+E229S+K360G+D458S+S582K+N672D+G753E+S754E+S757D+A769D+L775A+D801G+A843P+K875T+N892Y+T915A+N1008D, E229S+N440K+S582K +A624E+S635E+N672D+G738L+G753E+S754E+S757D+A769D+L775A+D801G+K875T +N892Y, S100D+E229S+K360G+D458S+S582K+N672D+G753E+S754E+A769D+L775A +D801G+K875T+N892Y+N1008D, A190Q+E229S+D458S+T631N+N672D+G753E+S754E+A769D+L775A+D801G+A843P+K875T+N892Y, A190Q+E229S+K360G+D458S+S582K+N672D+G753E+S754E+A769D+L775A+D801G+K875T+N892Y+N1008D, E229S +S635E+T649K+I656V+N672D+G753E+S754R+S757D+A769D+L775A+D801G+A843P +K875T+N892Y, E229S+D458S+S582K+T631N+S635E+N672D+M728V+G753E+S754E +S757D+A769D+L775A+D801G+K875T+N892Y, A190Q+E229S+K360G+D458S+S582K +N672D+G753E+S754E+S757D+A769D+L775A+D801G+K875T+N892Y+N1008D, E229S+A492L+S635E+T649K+I656V+N672D+G753E+S757D+A769D+L775A+D801G+K875T+N892Y, S100D+A190Q+E229S+K360G+D458S+S582K+N672D+G753E+S754E+A769D+L775A+D801G+K875T+N892Y+N1008D, A190Q+E229S+I234V+S582K+N672D+G753E+S754E+S757D+A769D+L775A+D801G+K875T+N892Y, E229S+N399K+D458S+A492H+K567R+S582K+S635E+T649K+N672D+G753E+S754E+A769D+L775A+D777R +D801G+K875T+N892Y, E229S+D458S+A492L+T631N+N672D+G753E+S754E+S757D +A769D+L775A+D801G+K875T+N892Y, E229S+D458S+A492H+K567R+S582K+S635E +N672D+G753E+S754E+A769D+L775A+D777R+D801G+K875T+N892Y, S100D+E229S +K360G+D458S+S582K+N672D+G753E+S754E+S757D+A769D+L775A+D801G+A843P+K875T+N892Y+N1008D, E229S+N399K+D458S+K567R+S582K+S635E+N672D +G753E+S754E+A769D+L775A+D777R+D801G+K875T+N892Y, preferably selected from the group consisting of E229N+N672D+P752K+G753E+A769D+L775A+D801G+K875T+N892Y, E229S+N672D+G753E+S754E+A769D+L775A+D801G+K875T+N892Y, E229S+N672D+P752R+G753E+S754E+A769D+L775A+D801G+K875T+N892Y, A190Q+E229S+I234V+A624E+N672D+G753E+S754E+A769D+L775A+D801G+K875T, A190Q+E229S+T631N+N672D+I703L+P752K+G753E+A769D+L775A+D801G+K875T, A190Q+E229S+I234V+S582K+N672D+G753E+S754E+S757D+A769D+L775A+D801G+K875T+N892Y, E229S+N440K+S582K+A624E+S635E+N672D+G738L+G753E+S754E+S757D+A769D +L775A+D801G+K875T+N892Y, and S100D+E229S+K360G+D458S+S582K+N672D+G753E+S754E+S757D+A769D+L775A+D801G+A843P+K875T+N892Y+N1008D, wherein said positions correspond to amino acid positions of SEQ ID NO:2.

In a particular aspect, the xanthan lyase variant has besides the aforementioned alterations no further alterations relative to the parent enzyme of SEQ ID NO:2, i.e. the remaining sequence is identical to SEQ ID NO:2.

In one aspect, the detergent composition comprises preferred endoglucanase variants in combination with preferred xanthan lyase variants.

In some aspects, the detergent composition thus comprises

(A) an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71 E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1; and/or

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D+L775A+D801G+K875T+N892Y, and (B8) S100D+E229S+K360G+D458S+S582K+N672D+G753E+S754E+S757D+A769D+L775A+D801G+A843P+K875T+N892Y +N1008D, wherein said positions correspond to amino acid positions of SEQ ID NO:2; and

(C) an enzyme stabilizing component.

In a particular aspect, detergent composition comprises

(A) an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71 E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1; and

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D+L775A+D801G+K875T+N892Y, and (B8) S100D+E229S+K360G+D458S+S582K+N672D+G753E+S754E+S757D+A769D+L775A+D801G+A843P+K875T+N892Y +N1008D, wherein said positions correspond to amino acid positions of SEQ ID NO:2; and

(C) an enzyme stabilizing component, wherein the enzyme stabilizing component is selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and sodium formate, more preferably being sodium formate.

In some aspects, the endoglucanase and/or the xanthan lyase variant do not comprise any further substitution besides those explicitly mentioned above, i.e. the remainder of the sequence is identical to that of the parent enzyme as set forth in SEQ ID NO:1 and SEQ ID NO:2, respectively.

In some aspects, the detergent composition comprises any one of the endoglucanase variants (A1), (A2), (A3), (A4), (A5), (A6), (A7), and (A8), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In some aspects, the detergent composition additionally comprises one or more further detergent components, preferably a surfactant.

In some aspects, the detergent composition comprises 0.000001 to 10 wt.-%, such as 0.00001 to 5 wt.-%, preferably 0.0001 to 1 wt.-%, more preferably 0.0005 to 0.5 wt.-%, even more preferably 0.001 to 0.1 wt.-%, of said endoglucanase variant (active enzyme per ml detergent composition) and/or 0.000001 to 10 wt.-%, such as 0.00001 to 5 wt.-%, preferably 0.0001 to 1 wt.-%, more preferably 0.0005 to 0.5 wt.-%, even more preferably 0.001 to 0.1 wt.-%, of said xanthan lyase variant (active enzyme per ml detergent composition) and/or 0.001 to 20 wt.-%, such as 0.005 to 15 wt.-%, preferably 0.01 to 10 wt.-%, more preferably 0.1 to 5.5 wt.-%, even more preferably 0.25 to 1.5 wt.-%, of said enzyme stabilizing agent (by weight of the detergent composition).

In some aspects, the detergent composition comprises 0.1 to 5.5 wt.-%, preferably 0.25 to 1.5 wt.-%, of sodium formate by weight of the detergent composition.

In a further aspect, the detergent composition comprises said endoglucanase variant and/or said xanthan lyase variant and said enzyme stabilizing agent in a weight ratio (enzyme:stabilizer) of 0.00001:1 to 0.1:2, preferably 0.0001:1 to 0.01:1, more preferably 0.0005:1 to 0.001:1.

In one aspect, the detergent composition comprises said endoglucanase variant and/or said xanthan lyase variant and sodium formate in a weight ratio (enzyme:sodium formate) of 0.0005:1 to 0.001:1, preferably 0.001:1 to 0.02:1, more preferably 0.005:1 to 0.01:1.

In a particular aspect, detergent composition thus comprises

(A) 0.0005 to 0.5 wt.-%, preferably 0.001 to 0.1 wt.-%, of an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1;

(B) 0.0005 to 0.5 wt.-%, preferably 0.001 to 0.1 wt.-%, of a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T +N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q +E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E +S754E+S757D+A769D+L775A+D801G+K875T+N892Y, and (B8) S100D+E229S+K360G +D458S+S582K+N672D+G753E+S754E+S757D+A769D+L775A+D801G+A843P+K875T +N892Y+N1008D, wherein said positions correspond to amino acid positions of SEQ ID NO:2;

(C) 0.1 to 5.5 wt.-%, preferably 0.25 to 1.5 wt.-%, of an enzyme stabilizing component, wherein the enzyme stabilizing component is selected from the group consisting of glycerol and formates, preferably being sodium formate; and

(D) 0 to 99 wt. % of at least one further detergent component, preferably a surfactant.

In some aspects, the present disclosure relates to use of a composition, wherein said use is selected from the group consisting of: use for degrading xanthan gum and use in a cleaning process, such as laundry or hard surface cleaning such as dish wash.

In some aspects, the present disclosure further relates to the use of a detergent composition for degrading xanthan gum, for washing or cleaning textiles and/or hard surfaces, such as dish wash, wherein the composition has an enzyme detergency benefit.

In some aspects, the present disclosure also relates to methods of degrading xanthan gum using detergent compositions, wherein xanthan gum is on the surface of a hard surface or textile.

In some aspects, the present disclosure relates to the use of an enzyme stabilizing component for improving stability of an endoglucanase variant and/or a xanthan lyase variant and/or a mixture thereof in a detergent composition, in particular in a liquid laundry detergent composition, wherein the endoglucanase variant and/or the xanthan lyase variant is/are as defined as above, and wherein the enzyme stabilizing component is selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and sodium formate, more preferably being sodium formate.

In a particular aspect, the present disclosure relates to the use of an enzyme stabilizing component for improving stability of an endoglucanase variant and/or a xanthan lyase variant and/or a mixture thereof in a detergent composition, wherein the endoglucanase variant has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprises a set of alterations selected from the group consisting of A559N+Y579W+T697G, K512P+A559N+Y579W+T697G, N18G+A71E +A186P+E408D+Y579W+I602T+A651P+A688G+V756Y, N18G+N189K+E408D+A559N+Y579W+A688G+T697G+V756Y+K921R+Y934G, S313D+E408D, R880K+N905D+K921R+Y934G, I302D+S313D+E408D+Y579W+I602T+A651P+T697G+R880K+K921R+Y934G, and N216Q+S313D+E408D+D476R+Y579W+I602T+F638N+A651P+T697G+W719R+R880K+T887K+K921R+Y934G, wherein said positions correspond to amino acid positions of SEQ ID NO:1; wherein the xanthan lyase variant has at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of E229N+N672D+P752K+G753E+A769D+L775A+D801G+K875T+N892Y, E229S+N672D+G753E+S754E+A769D+L775A+D801G+K875T+N892Y, E229S+N672D+P752R+G753E+S754E+A769D+L775A+D801G+K875T+N892Y, A190Q+E229S+I234V+A624E+N672D+G753E+S754E+A769D+L775A+D801G+K875T, A190Q+E229S+T631N+N672D+I703L+P752K+G753E+A769D+L775A+D801G+K875T, A190Q+E229S+I234V+S582K+N672D+G753E+S754E+S757D+A769D+L775A+D801G+K875T+N892Y, E229S+N440K+S582K+A624E+S635E+N672D+G738L+G753E+S754E+S757D+A769D+L775A+D801G+K875T+N892Y, and S100D+E229S+K360G+D458S+S582K+N672D+G753E+S754E+S757D+A769D+L775A+D801G+A843P+K875T+N892Y+N1008D, wherein said positions correspond to amino acid positions of SEQ ID NO:2; and wherein the enzyme stabilizing component is selected from the group of glycerol and sodium formate.

### DETAILED DESCRIPTION

### Overview of Sequence Listing

SEQ ID NO:1 is the amino acid sequence of the parent mature endoglucanase from a strain of Paenibacillus sp.

SEQ ID NO:2 is the amino acid sequence of the parent mature xanthan lyase from a strain of Paenibacillus sp.

### DEFINITIONS

Cleaning or detergent application: the term “cleaning or detergent application” means applying the endoglucanase variant and/or the xanthan lyase variant in any composition for the purpose of cleaning or washing, by hand, machine or automated, on a hard surface or a textile.

Color clarification: During washing and wearing loose or broken fibers can accumulate on the surface of the fabrics. One consequence can be that the colors of the fabric appear less bright or less intense because of the surface contaminations. Removal of the loose or broken fibers from the textile will partly restore the original colors and looks of the textile. By the term “color clarification”, as used herein, is meant the partial restoration of the initial colors of textile.

Corresponding to: The term “corresponding to” as used herein, refers to a way of determining the specific amino acid of a sequence wherein reference is made to a specific amino acid sequence. For example, when references are made to specific amino acid positions, the skilled person would be able to align another amino acid sequence to said amino acid sequence that reference has been made to, in order to determine which specific amino acid may be of interest in said another amino acid sequence. Alignment of another amino acid sequence with e.g. the sequence as set forth in SEQ ID NO:1 or 2 has been described elsewhere herein. Alternative alignment methods may be used and are well-known for the skilled person.

Degrading xanthan gum and xanthan gum degrading activity: The terms “degrading xanthan gum” and “xanthan gum degrading activity” are used interchangeably and are defined as the depolymerization, degradation or breaking down of xanthan gum into smaller components. The degradation of xanthan gum can either be the removal of one or more side chain saccharides, the cutting of the backbone of xanthan gum into smaller components or the removal of one or more side chain saccharides and the cutting of the backbone of xanthan gum into smaller components. A preferred assay for measuring degradation of xanthan gum is the reducing sugar assay as described in examples 2 and 3 herein. Non-limiting examples of the xanthan gum degrading activity include endoglucanase EC 3.2.1.4 activity and/or xanthan lyase EC 4.2.2.12 activity.

Detergent component: the term “detergent component” is defined herein to mean the types of chemicals which can be used in detergent compositions. Examples of detergent components are surfactants, hydrotropes, builders, cobuilders, chelators or chelating agents, bleaching system or bleach components, polymers, fabric hueing agents, fabric conditioners, foam boosters, suds suppressors, dispersants, dye transfer inhibitors, fluorescent whitening agents, perfume, optical brighteners, bactericides, fungicides, soil suspending agents, soil release polymers, anti-redeposition agents, enzyme inhibitors or stabilizers, enzyme activators, antioxidants, and solubilizers. The detergent composition may comprise of one or more of any type of detergent component.

Detergent composition: the term “detergent composition” (or “cleaning composition”) refers to compositions that find use in the removal of undesired compounds from items to be cleaned, such as textiles, dishes, and hard surfaces. The detergent composition may be used to e.g. clean textiles, dishes and hard surfaces for both household cleaning and industrial cleaning. The terms encompass any materials/compounds selected for the particular type of cleaning composition desired and the form of the product (e.g. liquid, gel, powder, granulate, paste, or spray compositions) and includes, but is not limited to, detergent compositions (e.g. liquid and/or solid laundry detergents and fine fabric detergents; hard surface cleaning formulations, such as for glass, wood, ceramic and metal counter tops and windows; carpet cleaners; oven cleaners; fabric fresheners; fabric softeners; and textile and laundry pre-spotters, as well as dish wash detergents). In addition to containing an endoglucanase and/or xanthan lyase, as described herein, the detergent formulation may contain one or more additional enzymes (such as proteases, amylases, lipases, cutinases, cellulases, endoglucanases, xyloglucanases, pectinases, pectin lyases, xanthanases, peroxidases, haloperoxygenases, oxidoreductases, catalases and mannanases, or any mixture thereof), and/or further detergent components.

Dish wash: The term “dish wash” refers to all forms of washing dishes, e.g. by hand or automatic dish wash. Washing dishes includes, but is not limited to, the cleaning of all forms of crockery such as plates, cups, glasses, bowls, all forms of cutlery such as spoons, knives, forks and serving utensils as well as ceramics, plastics, metals, china, glass and acrylics.

Dish washing composition: The term “dish washing composition” refers to all forms of compositions for cleaning hard surfaces. The present disclosure is not restricted to any particular type of dish wash composition or any particular detergent.

Endoglucanase: The term “endoglucanase” or “EG” means an endo-1,4-(1,3;1,4)-β-D-glucan 4-glucanohydrolase (EC 3.2.1.4) that catalyzes endohydrolysis of 1,4-β-D-glycosidic linkages in cellulose, cellulose derivatives (such as carboxymethyl cellulose and hydroxyethyl cellulose), lichenin, β-1,4 bonds in mixed β-1,3 glucans such as cereal β-D-glucans, xyloglucans, xanthans and other plant material containing cellulosic components. A preferred assay for measuring endoglucanase activity is the reducing sugar assay as described in example 2 herein. Non-limiting examples of endoglucanases include the mature parent endoglucanase having SEQ ID NO:1.

Endoglucanase variant having activity on xanthan gum pre-treated with xanthan lyase: The term “endoglucanase variant having activity on xanthan gum pre-treated with xanthan lyase” or an “endoglucanase having activity on xanthan gum pre-treated with xanthan lyase and belonging to the GH9 class of glycosyl hydrolases” is defined as a polypeptide comprising a domain belonging to the GH9 class of glycosyl hydrolases, and having activity (e.g. enzymatic activity, xanthan degrading activity, endoglucanase EC 3.2.1.4 activity) on xanthan gum pre-treated with xanthan lyase. A preferred assay for measuring activity on xanthan gum pre-treated with xanthan lyase is disclosed in example 2 herein.

Enzyme detergency benefit: The term “enzyme detergency benefit” is defined herein as the advantageous effect an enzyme may add to a detergent compared to the same detergent without the enzyme. Important detergency benefits which can be provided by enzymes are stain removal with no or very little visible soils after washing and or cleaning, prevention or reduction of redeposition of soils released in the washing process (an effect that also is termed anti-redeposition), restoring fully or partly the whiteness of textiles, which originally were white but after repeated use and wash have obtained a greyish or yellowish appearance (an effect that also is termed whitening). Textile care benefits, which are not directly related to catalytic stain removal or prevention of redeposition of soils are also important for enzyme detergency benefits. Examples of such textile care benefits are prevention or reduction of dye transfer from one fabric to another fabric or another part of the same fabric (an effect that is also termed dye transfer inhibition or anti-backstaining), removal of protruding or broken fibers from a fabric surface to decrease pilling tendencies or remove already existing pills or fuzz (an effect that also is termed anti-pilling), improvement of the fabric softness, color clarification of the fabric and removal of particulate soils which are trapped in the fibers of the fabric or garment. Enzymatic bleaching is a further enzyme detergency benefit where the catalytic activity generally is used to catalyze the formation of bleaching component such as hydrogen peroxide or other peroxides.

Hard surface cleaning: The term “hard surface cleaning” is defined herein as cleaning of hard surfaces wherein hard surfaces may include floors, tables, walls, roofs etc. as well as surfaces of hard objects such as cars (car wash) and dishes (dish wash).

Improved property: The term “improved property” means a characteristic associated with a variant that is improved compared to the parent. Such improved properties include, but are not limited to, catalytic efficiency, catalytic rate, chemical stability, oxidation stability, pH activity, pH stability, specific activity, stability under storage conditions, chelator stability, substrate binding, substrate cleavage, substrate specificity, substrate stability, surface properties, thermal activity, thermostability, wash performance, or stain removal.

Laundering: The term “laundering” relates to both household laundering and industrial laundering and means the process of treating textiles with a solution containing a cleaning or detergent composition. The laundering process can be carried out using e.g. a household or an industrial washing machine or can be carried out by hand.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 1 to 1055 of SEQ ID NO:1 or amino acids 1 to 1037 of SEQ ID NO:2.

Parent: The term “parent” or “parent endoglucanase” or “parent xanthan lyase” means any polypeptide with endoglucanase resp. xanthan lyase activity to which an alteration is made to produce the enzyme variants. In one aspect, the parent is an endoglucanase (resp. xanthan lyase) having the identical amino acid sequence of the variant, but not having the alterations at one or more of the specified positions. It will be understood that the expression “having identical amino acid sequence” relates to 100% sequence identity. Non-limiting examples of parent endoglucanases include the mature parent endoglucanase having SEQ ID NO:1. Non-limiting examples of parent xanthan lyases include the mature parent xanthan lyase having SEQ ID NO:2.

Sequence identity: The relatedness between two amino acid sequences is described by the parameter “sequence identity”. The sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the—nobrief option) is used as the percent identity and is calculated as follows: (Identical Residues×100)/(Length of Alignment−Total Number of Gaps in Alignment).

Stability: The term “stability” means resistance or the degree of resistance to change, unfolding, disintegration, denaturation or activity loss. Non-limiting examples of stability include conformational stability, storage stability and stability during use, e.g. during a wash process and reflects the stability of a polypeptide (e.g. an endoglucanase variant or xanthan lyase variant) as a function of time, e.g. how much activity is retained when said polypeptide (e.g. said endoglucanase variant or xanthan lyase variant) is kept in solution, in particular in a detergent solution. The stability is influenced by many factors, e.g. presence of chelators, pH, temperature, detergent composition, e.g. amount of builders, surfactants, chelators etc. The endoglucanase or xanthan lyase stability may be measured as described in examples 2 and 3 herein.

Textile: The term “textile” means any textile material including yarns, yarn intermediates, fibers, non-woven materials, natural materials, synthetic materials, and any other textile material, fabrics made of these materials and products made from fabrics (e.g. garments and other articles). The textile or fabric may be in the form of knits, wovens, denims, non-wovens, felts, yarns, and toweling. The textile may be cellulose based such as natural cellulosic, including cotton, flax/linen, jute, ramie, sisal or coir or manmade cellulosic (e.g. originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabbit and silk or synthetic polymer such as nylon, aramid, polyester, acrylic, polypropylene and spandex/elastane, or blends thereof as well as blend of cellulose based and non-cellulose based fibers. Examples of blends are blends of cotton and/or rayon/viscose with one or more companion material such as wool, synthetic fibers (e.g. polyamide fibers, acrylic fibers, polyester fibers, polyvinyl alcohol fibers, polyvinyl chloride fibers, polyurethane fibers, polyurea fibers, aramid fibers), and cellulose-containing fibers (e.g. rayon/viscose, ramie, flax/linen, jute, cellulose acetate fibers, lyocell). Fabric may be conventional washable laundry, e.g. stained household laundry. When the term fabric or garment is used it is intended to include the broader term textiles as well.

Variant: The term “variant” means a polypeptide (e.g. an endoglucanase polypeptide or a xanthan lyase polypeptide) comprising an alteration, i.e. a substitution, insertion, and/or deletion, at one or more (e.g. several) positions. A substitution means replacement of the amino acid occupying a position with a different amino acid; a deletion means removal of the amino acid occupying a position; and an insertion means adding one or more (e.g. several) amino acids, e.g. 1-5 amino acids, adjacent to and immediately following the amino acid occupying a position. Non-limiting examples of endoglucanase variants and/or xanthan lyase variants, as described herein, include endoglucanase variants and/or xanthan lyase variants having an activity on xanthan gum (for xanthan lyase) and xanthan gum pre-treated with xanthan lyase (for endoglucanase). Non-limiting examples of variants described herein further include variants having at least 20%, e.g. at least 40%, 50%, 60%, 70%, 80%, 90%, 95%, or 100% endoglucanase activity of the mature parent having SEQ ID NO:1 or SEQ ID NO:2. A preferred assay for measuring activity on xanthan gum (optionally pre-treated with xanthan lyase) is disclosed in examples 2 and 3 herein.

Conventions for designation of variants: The mature polypeptide disclosed in SEQ ID NO:1 is used to determine the corresponding amino acid residue in another endoglucanase and the mature polypeptide disclosed in SEQ ID NO:2 is used to determine the corresponding amino acid residue in another xanthan lyase. The amino acid sequence of another endoglucanase/xanthan lyase is aligned with the mature polypeptide disclosed in SEQ ID NO:1 or, and based on the alignment, the amino acid position number corresponding to any amino acid residue in the mature polypeptide disclosed in SEQ ID NO:1 or 2 is determined using the Needleman-Wunsch algorithm (as described above). Identification of the corresponding amino acid residue in another endoglucanase or xanthan lyase can be determined by an alignment of multiple polypeptide sequences using several computer programs, which are known to the skilled person. In describing the variants, the nomenclature described below is adapted for ease of reference. The accepted IUPAC single letter or three letter amino acid abbreviation is employed.

Substitutions: For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g. “Gly205Arg+Ser411Phe” or “G205R+S411F”, representing substitutions at positions 205 and 411 of glycine (G) with arginine (R) and serine (S) with phenylalanine (F), respectively.

Deletions: For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g. “Gly195*+Ser411*” or “G195*+S411*”.

Insertions: For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”. An indication of an insertion at a particular position is understood as being an insertion after the original amino acid residue. For example, an “insertion at position 195” is understood to be an insertion after the original residue in position 195.

In such cases the inserted amino acid residue(s) are numbered by the addition of lower-case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations: Variants comprising multiple alterations are separated by addition marks (“+”), e.g. “Arg170Tyr+Gly195Glu” or “R170Y+G195E” representing a substitution of arginine and glycine at positions 170 and 195 with tyrosine and glutamic acid, respectively.

Different alterations: Where different alterations can be introduced at a position, the different alterations are separated by a comma, e.g. “Arg170Tyr,Glu” represents a substitution of arginine at position 170 with tyrosine or glutamic acid. Thus, “Tyr167Gly,Ala+Arg170Gly,Ala” designates the following variants: “Tyr167Gly+Arg170Gly”, “Tyr167Gly+Arg170Ala”, “Tyr167Ala+Arg170Gly”, and “Tyr167Ala+Arg170Ala”. Alternatively, different alterations or may be indicated using brackets, e.g. Arg170[Tyr, Gly] or in one-letter code R170[Y,G].

Wash performance: The term “wash performance” is used as an enzyme's ability to remove stains present on the object to be cleaned during e.g. wash or hard surface cleaning. The improvement in the wash performance may be quantified by calculating the so-called intensity value (Int) in ‘Automatic Mechanical Stress Assay (AMSA) for laundry’ or the remission value (Rem).

Xanthan lyase variant having activity on xanthan gum: The term “xanthan lyase variant having activity on xanthan gum” is defined as a polypeptide that cleaves the β-D-mannosyl-β-D-1,4-glucuronosyl bond of xanthan (e.g. xanthan lyase EC 4.2.2.12 activity). A preferred assay for measuring activity on xanthan gum is disclosed in example 3 herein. Examples of the xanthan lyase variants having activity on xanthan gum, are xanthan lyase polypeptides as such. Thus, polypeptides that that cleaves the β-D-mannosyl-β-D-1,4-glucuronosyl bond of xanthan.

### DETAILED DESCRIPTION

The known xanthan endoglucanase having SEQ ID NO:1 and the xanthan lyase having SEQ ID NO:2 are both large enzymes (>1000 amino acid residues). It is therefore extremely laborious and expensive to target its properties for improvement of, e.g. stability in a detergent composition, e.g. in the presence of a surfactant or a chelator.

The inventors have surprisingly found that the stability of specific endoglucanase variants and/or xanthan lyase variants in detergent compositions can be improved by addition of certain enzyme stabilizing components, like e.g. being selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and formates, in particular by addition of sodium formate.

In one embodiment, the detergent composition comprises an endoglucanase variant comprising an alteration, preferably a substitution, at one or more positions in one or more regions selected from the group consisting of region 1 corresponding to amino acids 95 to 105 of SEQ ID NO:1, region 2 corresponding to amino acids 115 to 138 of SEQ ID NO:1, region 3 corresponding to amino acids 210 to 251 of SEQ ID NO:1, region 4 corresponding to amino acids 267 to 301 of SEQ ID NO:1, region 5 corresponding to amino acids 339 to 361 of SEQ ID NO:1, region 6 corresponding to amino acids 547 to 595 of SEQ ID NO:1, region 7 corresponding to amino acids 612 to 660 of SEQ ID NO:1, region 8 corresponding to amino acids 806 to 828 of SEQ ID NO:1, region 9 corresponding to amino acids 839 to 1042 of SEQ ID NO:1, region 10 corresponding to amino acids 1 to 94 of SEQ ID NO:1, region 11 corresponding to amino acids 106 to 114 of SEQ ID NO:1, region 12 corresponding to amino acids 139 to 209 of SEQ ID NO:1, region 13 corresponding to amino acids 252 to 266 of SEQ ID NO:1, region 14 corresponding to amino acids 302 to 338 of SEQ ID NO:1, region 15 corresponding to amino acids 362 to 546 of SEQ ID NO:1, region 16 corresponding to amino acids 596 to 611 of SEQ ID NO:1, region 17 corresponding to amino acids 661 to 805 of SEQ ID NO:1, region 18 corresponding to amino acids 829 to 838 of SEQ ID NO:1, and region 19 corresponding to amino acids 1043 to 1055 of SEQ ID NO:1.

In one embodiment, the detergent composition comprises a xanthan lyase variant comprising an alteration, preferably a substitution, at one or more positions in one or more regions selected from the group consisting of region 1 corresponding to amino acids 154 to 176 of SEQ ID NO:2, region 2 corresponding to amino acids 614 to 658 of SEQ ID NO:2, region 3 corresponding to amino acids 731 to 803 of SEQ ID NO:2, region 4 corresponding to amino acids 807 to 846 of SEQ ID NO:2, region 5 corresponding to amino acids 872 to 885 of SEQ ID NO:2, region 6 corresponding to amino acids 903 to 1004 of SEQ ID NO:2, region 7 corresponding to amino acids 1 to 153 of SEQ ID NO:2, region 8 corresponding to amino acids 177 to 613 of SEQ ID NO:2, region 9 corresponding to amino acids 659 to 730 of SEQ ID NO:2, region 10 corresponding to amino acids 804 to 806 of SEQ ID NO:2, region 11 corresponding to amino acids 847 to 871 of SEQ ID NO:2, region 12 corresponding to amino acids 886 to 902 of SEQ ID NO:2, and region 13 corresponding to amino acids 1005 to 1037 of SEQ ID NO:2.

In one embodiment, the present disclosure provides detergent compositions comprising variants of an endoglucanase and of a xanthan lyase, as described herein, both of which have significantly improved stability in said detergent composition due to addition of an enzyme stabilizing component.

In one embodiment, the enzyme stabilizing component is selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and formates.

In a preferred embodiment, the enzyme stabilizing component is glycerol and/or sodium formate.

Endoglucanase Variants

In one embodiment, the detergent composition comprises an endoglucanase variant comprising an alteration, preferably a substitution, at one or more positions in one or more regions selected from the group consisting of region 1 corresponding to amino acids 95 to 105 of SEQ ID NO:1, region 2 corresponding to amino acids 115 to 138 of SEQ ID NO:1, region 3 corresponding to amino acids 210 to 251 of SEQ ID NO:1, region 4 corresponding to amino acids 267 to 301 of SEQ ID NO:1, region 5 corresponding to amino acids 339 to 361 of SEQ ID NO:1, region 6 corresponding to amino acids 547 to 595 of SEQ ID NO:1, region 7 corresponding to amino acids 612 to 660 of SEQ ID NO:1, region 8 corresponding to amino acids 806 to 828 of SEQ ID NO:1, region 9 corresponding to amino acids 839 to 1042 of SEQ ID NO:1, region 10 corresponding to amino acids 1 to 94 of SEQ ID NO:1, region 11 corresponding to amino acids 106 to 114 of SEQ ID NO:1, region 12 corresponding to amino acids 139 to 209 of SEQ ID NO:1, region 13 corresponding to amino acids 252 to 266 of SEQ ID NO:1, region 14 corresponding to amino acids 302 to 338 of SEQ ID NO:1, region 15 corresponding to amino acids 362 to 546 of SEQ ID NO:1, region 16 corresponding to amino acids 596 to 611 of SEQ ID NO:1, region 17 corresponding to amino acids 661 to 805 of SEQ ID NO:1, region 18 corresponding to amino acids 829 to 838 of SEQ ID NO:1, and region 19 corresponding to amino acids 1043 to 1055 of SEQ ID NO:1.

In one embodiment, such variant has at least 60% and less than 100% sequence identity to SEQ ID NO:1, preferably said endoglucanase variant has activity on xanthan gum pre-treated with xanthan lyase, further preferably said activity is a xanthan gum degrading activity.

In one embodiment, the detergent composition comprises an endoglucanase variant, comprising an alteration, preferably a substitution, at one or more positions in one or more regions or multiple alterations (such as 2, 3, 4, 5, 6, 7, 8, 9 or 10) in one region or multiple alterations (e.g. 2, 3, 4, 5, 6, 7, 8, 9 or 10) in multiple regions (e.g. 2, 3, 4, 5, 6, 7, 8, or 9) selected from the group consisting of region 1 corresponding to amino acids 95 to 105 of SEQ ID NO:1, region 2 corresponding to amino acids 115 to 138 of SEQ ID NO:1, region 3 corresponding to amino acids 210 to 251 of SEQ ID NO:1, region 4 corresponding to amino acids 267 to 301 of SEQ ID NO:1, region 5 corresponding to amino acids 339 to 361 of SEQ ID NO:1, region 6 corresponding to amino acids 547 to 595 of SEQ ID NO:1, region 7 corresponding to amino acids 612 to 660 of SEQ ID NO:1, region 8 corresponding to amino acids 806 to 828 of SEQ ID NO:1, region 9 corresponding to amino acids 839 to 1042 of SEQ ID NO:1, region 10 corresponding to amino acids 1 to 94 of SEQ ID NO:1, region 11 corresponding to amino acids 106 to 114 of SEQ ID NO:1, region 12 corresponding to amino acids 139 to 209 of SEQ ID NO:1, region 13 corresponding to amino acids 252 to 266 of SEQ ID NO:1, region 14 corresponding to amino acids 302 to 338 of SEQ ID NO:1, region 15 corresponding to amino acids 362 to 546 of SEQ ID NO:1, region 16 corresponding to amino acids 596 to 611 of SEQ ID NO:1, region 17 corresponding to amino acids 661 to 805 of SEQ ID NO:1, region 18 corresponding to amino acids 829 to 838 of SEQ ID NO:1, and region 19 corresponding to amino acids 1043 to 1055 of SEQ ID NO:1, wherein said variant has at least 60% and less than 100% sequence identity to SEQ ID NO:1, preferably said endoglucanase variant has activity on xanthan gum pre-treated with xanthan lyase, further preferably said activity is a xanthan gum degrading activity.

In one embodiment, the detergent composition comprises an endoglucanase variant comprising an alteration, preferably a substitution, at one or more positions in: (i) regions 6 and 17; (ii) regions 6, 15 and 17; (iii) regions 10, 12 and 15; (iv) regions 6, 7, 16, and 17; (v) region 6, 9, 10, 12, 15, and 17; (vi) region 14 and 15; (vii) region 9; (viii) 6, 7, 9, 14, 15, 16, and 17; or (ix) 3, 6, 7, 9, 14, 15, 16, and 17; wherein said variant preferably has no alternation in the other regions besides those mentioned.

In one embodiment, the detergent composition comprises an endoglucanase variant having at least 60%, e.g. at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and less than 100% sequence identity to the amino acid sequence as set forth in SEQ ID NO:1.

In one embodiment, the detergent composition comprises an endoglucanase variant comprising an alteration, preferably a substitution, at one or more positions selected from the group consisting of 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 719, 720, 744, 749, 754, 756, 760, 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045 and 1048, wherein said positions correspond to amino acid positions of SEQ ID NO:1.

In one embodiment, the detergent composition comprises an endoglucanase variant comprising an alteration, preferably a substitution, at 2, 3, 4, 5, 6, 7, 8, 9 or more positions selected from the group consisting of 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 719, 720, 744, 749, 754, 756, 760, 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045 and 1048, wherein said positions correspond to amino acid positions of SEQ ID NO:1.

In a preferred embodiment, the detergent composition comprises an endoglucanase variant comprising an alteration, preferably a substitution, at one or more positions selected from the group consisting of 17, 20, 216, 283, 302, 311, 313, 408, 476, 579, 602, 627, 636, 638, 651, 688, 697, 719, 756, 880, 881, 883, 887, 906, 921, 934, 937, and 956, wherein said positions correspond to amino acid positions of SEQ ID NO:1.

In a more preferred embodiment, the detergent composition comprises an endoglucanase variant comprising an alteration, preferably a substitution, at each position (or at least four positions) corresponding to positions 17, 20, 216, 283, 302, 311, 313, 408, 476, 579, 602, 627, 636, 638, 651, 688, 697, 719, 756, 880, 881, 883, 887, 906, 921, 934, 937, and 956, wherein said positions correspond to amino acid positions of SEQ ID NO:1.

In one embodiment, the detergent composition comprises an endoglucanase variant comprising one or more amino acid substitutions selected from the group consisting of V4T, S17A, N18G, F20P, F20N, F20G, F20Y, K51Q, K51H, E53Y, E53P, E53G, Y55M, Y55D, V56M, Y60F, S63F, A71E, T87R, T92S, K99R, A120P, I125V, A126R, S128X, N129D, K130R, F137L, H182Y, A186P, N189K, K192N, K213R, N216D, N216Q, N216R, A221R, L226K, K228E, K228I, G230F, G230L, G230A, G230H, G230N, G230W, G230T, F231Y, F231N, V232R, V232G, L233H, H235D, N240Q, G243K, G243R, D247N, A249N, A278S, G279E, K281F, K281V, K281Y, K281H, K281Q, K281N, K281W, K281T, K281R, A283D, N285G, N285L, N285M, N285S, N285P, N285T, N285Y, N285H, N285K, N285D, N285W, N285R, Q289E, T292A, T292F, T292L, T292I, T292V, T292S, T292P, T292Y, T292Q, T292N, T292K, T292D, T292G, A294V, F297L, Q298E, I302D, I302H, I302V, I302M, H311N, S313D, W333L, A346H, A346D, T353D, A386P, I387T, K388R, K390Q, I403Y, E408D, E408N, E408S, E408P, E408A, E408G, P410G, Q416S, Q416D, N441G, A448E, A448W, A448S, K451Q, K451S, G471S, S472Y, D476R, Q489P, K507R, K512P, S515V, S538C, L555Q, G556S, G557R, N558P, N558F, N558K, N558D, N558M, N558Q, N558I, N558Y, N558H, N558E, A559N, A559F, A559M, A559P, A559Y, A559H, A559Q, A559D, A559R, A559G, A559I, A559S, A559K, S560F, S560P, S560K, S560G, S560D, T561E, T561Q, T561S, T561D, T561P, G562W, G562P, G563E, A564I, A564Y, A564H, A564Q, A564K, A564E, A564D, E565M, V567F, V567P, K568R, L569F, L569Y, L569D, L569E, P570F, P570L, P570I, P570M, P570V, P570S, P570T, P570A, P570Y, P570H, P570Q, P570N, P570K, P570E, P570W, P570R, P570G, I575V, I575M, I575A, I575D, I575E, I576F, I576M, I576P, D578R, Y579F, Y579W, V580L, T581M, D583M, Q589G, P590S, P590T, P590E, E591L, G592D, S593P, S593H, S593Q, S593N, S593K, S593D, S593E, S593R, S595L, S598Q, A599S, I602T, I602D, V603P, S605T, S607C, G609E, S616G, S616D, K627L, K627M, K627V, K627S, K627T, K627Q, K627R, I630F, I630V, I630Y, K631R, K631A, T633V, D635L, D635M, D635T, D635A, D635P, D635N, D635K, D635E, D635G, D635W, S636L, S636M, S636A, S636H, S636Q, S636N, S636K, S636R, F638N, F638Y, T639D, F638I, F638V, F638T, F638L, F638H, F638M, T639V, T639S, T639L, T639I, T639M, T639A, T639E, T639W, T639G, T639Y, T639P, T640S, Y641E, S642T, S642N, N643D, N643H, N643T, T644F, A651P, A651S, D676H, Q683E, A688G, Y690F, T694A, T697G, R698W, T699A, T706Q, T711S, T711V, T711Y, K713R, W719R, K720H, K744H, K744Q, A749T, K754R, V756Y, V756H, S760G, T781M, N786K, T797S, S810Q, S810R, A811S, V812F, V812I, V812M, V812W, V812R, N815V, N815Y, N815E, N815W, N815R, S823Q, A824T, A824D, T825G, T825N, T825W, T825A, T825D, V827I, V827M, V827S, N828D, N833D, Q834E, S835A, S835D, V837I, T843V, N848D, A868E, A869V, D870F, D870L, D870I, D870M, D870V, D870S, D870T, D870Y, D870H, D870Q, D870N, D870K, D870E, D870W, D870R, D870G, P871F, P871L, P871I, P871M, P871V, P871S, P871T, P871A, P871Y, P871H, P871Q, T872S, T872F, T872A, T872Y, T872H, T872Q, T872N, T872K, T872D, T872E, T872W, T872R, T872G, D873K, D873E, T874V, T874S, T874P, T874A, T874H, T874Q, T874N, T874K, R880K, V881Q, V881T, T883R, T883V, T883C, T883K, Y884H, A885F, A885Q, A885N, T887L, T887I, T887S, T887H, T887R, T887K, L888M, V890R, T892P, T892V, K894E, R898Q, N905D, F906A, Q912V, N920P, N920D, K921R, K921E, K921D, A924D, V926F, V926P, K927R, S928D, T932A, N933V, N933S, Y934G, Y934M, Y934S, Y934A, Y934Q, Y934N, Y934E, Y934W, Y934R, T935W, A937F, A937V, A937S, A937T, A937Q, A937D, A937E, V938I, K939I, K939V, D940E, N941S, N941H, N941D, A942P, A942E, D943Y, D943H, G946R, K948R, K948E, R950V, R950H, R950N, F952S, F952W, N953Y, G954L, Q956Y, Q956S, A957L, A957P, Y960F, A964N, A964C, N966P, N966C, G971A, T972K, Q974K, Q974C, M980I, Q989I, Q991L, Q991I, Q991M, Q991V, Q991T, Q991K, Q991C, G994D, G994N, S995I, S995V, S995Q, S995R, S995C, G998V, G998A, T999R, S1006T, S1006A, S1006K, S1006R, K1009E, Y1010W, L1011M, L1011S, L1011A, L1011Q, L1011N, L1011D, L1011E, R1029N, F1030M, K1031I, K1031S, K1031T, K1031H, V1032G, K1035A, A1037E, A1037W, S1038L, S1038I, S1038G, L1040N, L1040E, G1041F, G1041R, Y1042N, L1044F, L1044S, L1044N, L1044W, P1045Q, P1045W, and F1048W, wherein said positions correspond to amino acid positions of SEQ ID NO:1.

In a preferred embodiment, the detergent composition comprises an endoglucanase variant comprising one or more amino acid substitutions selected from the group consisting of S17A, F20P, N216D, N216Q, A283D, I302D, H311N, S313D, E408D, D476R, Y579W, I602T, K627R, S636N, S636K F638I, F638N, A651P, A688G, T697G, W719R, V756Y, R880K, V881Q, T883R, T887K, F906A, K921R, Y934G, A937E, and Q956Y, wherein said positions correspond to amino acid positions of SEQ ID NO:1.

In a further preferred embodiment, the detergent composition comprises an endoglucanase variant comprising a set of alterations selected from the group consisting of A559K+N558K+S560F+T561P+G562W, A559N+P570Q, A559N+P570T, A559N+Y579F, A559N+Y579F+K627R, A559N+Y579W, A559N+Y579W+K99R, A559N+Y579W+S128X+K627R, A559N+Y579W+S128X+S636N, A559N+Y579W+K281R, A559N+Y579W+Q416D+S636N, A559N+Y579W+S560P, A559N+Y579W+A564I, A559N+Y579W+P570N, A559N+Y579W+P570K, A559N+Y579W+P570R, A559N+Y579W+P570A, A559N+Y579W+P570T, A559N+Y579W+P570S, A559N+Y579W+P570Q, A559N+Y579W+P570H, A559N+Y579W+S616D, A559N+Y579W+S616D+K627R, A559N+Y579W+S616D+S636K, A559N+Y579W+S616D+A651P, A559N+Y579W+S616D+K921R, A559N+Y579W+S616D+K921R+Y934G, A559N+Y579W+K627R, A559N+Y579W+K627R+S636N+A651P, A559N+Y579W+K627R+S636N+K921R, A559N+Y579W+K627R+K921R, A559N+Y579W+K627M, A559N+Y579W+K627M+A651P, A559N+Y579W+S636N, A559N+Y579W+S636N+A651P, A559N+Y579W+S636N+K921R, A559N+Y579W+S636K+A651P, A559N+Y579W+S636K+K921R, A559N+Y579W+S636L, A559N+Y579W+F638I, A559N+Y579W+A651P, A559N+Y579W+A651P+K921R+Y934G, A559N+Y579W+A651P+Y934G, A559N+Y579W+D870M, A559N+Y579W+K921R, A559N+Y579W+K921R+A651P, A559N+Y579W+K921R+Y934G, A559N+Y579W+Y934G, A559N+Y579W+K948E, A559N+Y579W+K1009E, A559N+K627R, Y579F+A564E, Y579W+K99R, Y579W+G562P, Y579W+A564D 579W+P570K, Y579W+P570Q, Y579W+K627R, Y579W+S636N, Y579W+F638I, Y579W+A651P, Y579W+K921R, Y579W+Y934G, Y579W+K948E, Y579W+K1009E, K627R+K51Q, K627R+K451S, K627R+P570Q, K627R+P570T, K627R+S636N, K627R+F638I, K627R+A885F, K627R+Y934G, S636N+P570T, S636N+K921R, S636N+Y934G, F638I+P570K, F638I+P570Q, F638I+P570T, F638I+A651P, F638I+Y934G, F638I+K921R, A651P+P570Q, A651P+P570T, A885F+P570T, A885F+Y934G, K921R+P570Q, K921R +P570T, Y934G+P570T, wherein said positions correspond to amino acid positions of SEQ ID NO:1.

In an even more preferred embodiment, the detergent composition comprises an endoglucanase variant comprising a set of alterations selected from the group consisting of 559N+Y579W+T697G, K512P+A559N+Y579W+T697G, N18G+A71E+A186P+E408D+Y579W+I602T+A651P+A688G+V756Y, N18G+N189K+E408D+A559N+Y579W+A688G+T697G+V756Y+K921R+Y934G, S313D+E408D, R880K+N905D+K921R+Y934G, I302D+S313D+E408D+Y579W+I602T+A651P+T697G+R880K+K921R+Y934G, and N216Q+S313D+E408D+D476R+Y579W+I602T+F638N+A651P+T697G+W719R+R880K+T887K+K921R+Y934G, wherein said positions correspond to amino acid positions of SEQ ID NO:1.

In a particular aspect, the endoglucanase variant has besides the aforementioned alterations no further alterations relative to the parent enzyme of SEQ ID NO:1, i.e. the remaining sequence is identical to SEQ ID NO:1.

Xanthan Lyase Variants

In one embodiment, the detergent composition comprises a xanthan lyase variant comprising an alteration, preferably a substitution, at one or more positions in one or more regions selected from the group consisting of region 1 corresponding to amino acids 154 to 176 of SEQ ID NO:2, region 2 corresponding to amino acids 614 to 658 of SEQ ID NO:2, region 3 corresponding to amino acids 731 to 803 of SEQ ID NO:2, region 4 corresponding to amino acids 807 to 846 of SEQ ID NO:2, region 5 corresponding to amino acids 872 to 885 of SEQ ID NO:2, region 6 corresponding to amino acids 903 to 1004 of SEQ ID NO:2, region 7 corresponding to amino acids 1 to 153 of SEQ ID NO:2, region 8 corresponding to amino acids 177 to 613 of SEQ ID NO:2, region 9 corresponding to amino acids 659 to 730 of SEQ ID NO:2, region 10 corresponding to amino acids 804 to 806 of SEQ ID NO:2, region 11 corresponding to amino acids 847 to 871 of SEQ ID NO:2, region 12 corresponding to amino acids 886 to 902 of SEQ ID NO:2, and region 13 corresponding to amino acids 1005 to 1037 of SEQ ID NO:2.

In one embodiment, such variant has at least 60% and less than 100% sequence identity to SEQ ID NO:2, preferably said xanthan lyase variant has activity on xanthan gum, further preferably said activity is a xanthan gum degrading activity.

In one embodiment, the detergent composition comprises a xanthan lyase variant, comprising an alteration, preferably a substitution, at one or more positions in one or more regions or multiple alterations (such as 2, 3, 4, 5, 6, 7, 8, 9 or 10) in one region or multiple alterations (e.g. 2, 3, 4, 5, 6, 7, 8, 9 or 10) in multiple regions (e.g. 2, 3, 4, 5, 6, 7, 8, or 9) selected from the group consisting of: region 1 corresponding to amino acids 154 to 176 of SEQ ID NO:2, region 2 corresponding to amino acids 614 to 658 of SEQ ID NO:2, region 3 corresponding to amino acids 731 to 803 of SEQ ID NO:2, region 4 corresponding to amino acids 807 to 846 of SEQ ID NO:2, region 5 corresponding to amino acids 872 to 885 of SEQ ID NO:2, region 6 corresponding to amino acids 903 to 1004 of SEQ ID NO:2, region 7 corresponding to amino acids 1 to 153 of SEQ ID NO:2, region 8 corresponding to amino acids 177 to 613 of SEQ ID NO:2, region 9 corresponding to amino acids 659 to 730 of SEQ ID NO:2, region 10 corresponding to amino acids 804 to 806 of SEQ ID NO:2, region 11 corresponding to amino acids 847 to 871 of SEQ ID NO:2, region 12 corresponding to amino acids 886 to 902 of SEQ ID NO:2, and region 13 corresponding to amino acids 1005 to 1037 of SEQ ID NO:2, wherein said variant has at least 60% and less than 100% sequence identity to SEQ ID NO:2, preferably said xanthan lyase variant has activity on xanthan gum, further preferably said activity is a xanthan gum degrading activity.

In one embodiment, the xanthan lyase variant, as described herein, is one that comprises an alteration, preferably a substitution, at one or more positions in: (i) regions 3 and 5; (ii) regions 3, 5 and 12; (iii) regions 8, and 9; (iv) regions 2, 3, and 5; (v) regions 2, 3, 5, and 12; (vi) regions 3, 5, 8, 9, and 12; (vii) regions 2, 3, 5, 8, and 9; (viii) 3, 5, 8, 9, and 12; (ix) 2, 3, 5, 8, 9, and 12; (x) region 3; (xi) regions 3, 4 and 5; (xii) regions 7, 8 and 9; (xiii) regions 12 and 13; (xiv) regions 3, 4, 5, 8, 9, and 12; (xv) regions 8, 9, 12, and 13; (xvi) regions 7, 8, 9, 12, and 13; (xvii) regions 3, 4, 5, 7, 8, 9, and 12; and (xviii) regions 3, 4, 5, 7, 8, 9, 12, and 13, wherein said variant preferably has no alteration in the other regions besides those mentioned.

In one embodiment, the detergent composition comprises a xanthan lyase variant having at least 60%, e.g. at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% and less than 100% sequence identity to the amino acid sequence as set forth in SEQ ID NO:2.

In one embodiment, the detergent composition comprises a xanthan lyase variant comprising an alteration, preferably a substitution, at one or more positions selected from the group consisting of 9, 15, 18, 46, 58, 66, 89, 95, 100, 106, 109, 155, 159, 183, 188, 190, 203, 204, 221, 229, 234, 238, 240, 242, 243, 257, 258, 284, 291, 293, 316, 317, 320, 324, 329, 333, 339, 341, 352, 354, 360, 372, 377, 399, 400, 419, 440, 450, 451, 454, 458, 481, 492, 505, 533, 567, 568, 576, 578, 579, 582, 620, 624, 626, 631, 635, 649, 650, 656, 664, 672, 703, 722, 726, 727, 728, 738, 745, 752, 753, 754, 757, 769, 775, 777, 779, 782, 786, 789, 792, 796, 799, 801, 819, 824, 843, 851, 855, 856, 867, 875, 887, 892, 899, 900, 901, 902, 903, 911, 912, 915, 919, 921, 923, 925, 927, 928, 930, 933, 941, 966, 991, 1008 and 1016, wherein said positions correspond to amino acid positions of SEQ ID NO:2.

In one embodiment, the detergent composition comprises a xanthan lyase variant comprising an alteration, preferably a substitution, at 2, 3, 4, 5, 6, 7, 8, 9 or more positions selected from the group consisting of 9, 15, 18, 46, 58, 66, 89, 95, 100, 106, 109, 155, 159, 183, 188, 190, 203, 204, 221, 229, 234, 238, 240, 242, 243, 257, 258, 284, 291, 293, 316, 317, 320, 324, 329, 333, 339, 341, 352, 354, 360, 372, 377, 399, 400, 419, 440, 450, 451, 454, 458, 481, 492, 505, 533, 567, 568, 576, 578, 579, 582, 620, 624, 626, 631, 635, 649, 650, 656, 664, 672, 703, 722, 726, 727, 728, 738, 745, 752, 753, 754, 757, 769, 775, 777, 779, 782, 786, 789, 792, 796, 799, 801, 819, 824, 843, 851, 855, 856, 867, 875, 887, 892, 899, 900, 901, 902, 903, 911, 912, 915, 919, 921, 923, 925, 927, 928, 930, 933, 941, 966, 991, 1008 and 1016, wherein said positions correspond to amino acid positions of SEQ ID NO:2.

In a preferred embodiment, the detergent composition comprises a xanthan lyase variant comprising an alteration at one or more positions selected from the group consisting of 624, 631, 635, 649, 656, 738, 752, 753, 754, 757, 769, 775, 777, 800, 801, 843, 875, 911 and 915, and/or an alteration at one or more positions selected from the group consisting of 89, 100, 190, 229, 234, 352, 360, 399, 440, 458, 492, 567, 582, 664, 672, 703, 728, 892, 1008 and 1016, wherein said positions correspond to amino acid positions of SEQ ID NO:2.

In one embodiment, the detergent composition comprises a xanthan lyase variant comprising one or more amino acid substitutions selected from the group consisting of K9R, N15T, T18D, L46D, A58L, S66H, Q89Y, K95E, S100D, N106Y, Q109R, Q109D, Q109F, Q109K, Q109A, Y155E, A159P, K183Q, K183R, V188I, A190Q, A203P, K204R, A221P, E229N, E229S, E229V, I234V, I238W, I238L, I238M, I240W, N242S, G243V, Y257W, R258E, R284G, K291R, A293G, A293P, K316R, R317K, K320R, L324Q, K329R, K333R, L339M, I341P, V352I, S354P, K360G, K360R, Q372H, F377Y, N399K, K400R, F419Y, N440K, D450P, K451E, K451R, A454V, D458S, K481R, A492H, A492L, T505I, L533I, K567R, G568A, S578K, S578N, S578R, S579R, S579K, S582K, K620R, A624E, A626Q, T631N, S635E, S635T, T649V, T649K, Q650G, I656V, T664K, N672D, I703L, I722F, P726Q, T727P, M728V, G738L, K745R, P752R, P752K, G753E, G753Q, G753S, S754E, S754L, S754Q, S754R, S757D, S757P, S757E, A769D, A769T, A769R, L775A, L775F, L775I, L775M, L775Q, L775S, L775Y, D777R, P779V, Y782I, N786K, G789R, K792W, K792Y, K792V, K792A, N796Q, A799H, D801G, K819R, K824R, A843P, S851F, K855R, E856D, P867S, K875T, K875E, K887R, N892Y, N892W, N892F, G899S, I900G, D901A, T902F, T903A, T903Q, A911M, A911V, A911S, A912T, A912I, A912Y, T915S, T915V, T915Q, T919D, T919F, T919G, T921R, T921S, T923D, T923H, T925D, T925Q, T925R, T927K, D928W, Y930F, Y930H, Y930L, D933M, G941D, G941E, A966P, N991D, V998K, N1008D and K1016T, wherein said positions correspond to amino acid positions of SEQ ID NO:2.

In a preferred embodiment, the detergent composition comprises a xanthan lyase variant comprising one or more substitutions selected from the group consisting of Q89Y, S100D, A190Q, E229S, I234V, V352I, K360G, N399K, N440K, D458S, A492H, A492L, K567R, S582K, T664K, N672D, I703L, M728V, N892Y N1008D and K1016T, and/or one or more substitutions selected from the group consisting of A624E, T631N, S635E, T649K, 1656V, G738L, P752K, P752R, G753E, S754E, S754R, S757D, A769D, L775A, D777R, V800P, D801G, A843P, K875T, A911V and T915A, wherein said positions correspond to amino acid positions of SEQ ID NO:2.

In one embodiment, the detergent composition comprises a xanthan lyase variant comprising a set of alterations selected from the group consisting of A190Q+E229S+S635E+T649K+I656V+N672D+I703L+G753E+S754E+S757D+A769D+L775A+D801G+K875T+N892Y+N1008D, E229S+S635E+T649K+I656V+N672D+I703L+G753E+S754E+A769D+L775A+D801G+K875T+N892Y+N1008D, E229S+V352I+S635E+T649K+I656V+N672D+G753E+S754E+A769D+L775A+V800P+D801G+K875T+N892Y, E229S+K360G+D458S+S582K+N672D+G753E+S754E+A769D+L775A+D801G+K875T+N892Y+N1008D, S100D+E229S+K360G +D458S+S582K+T664K+N672D+G753E+S754E+S757D+A769D+L775A+D801G+A843P+K875T+N892Y+A911V+N1008D+K1016T, E229S+I234V+S582K+N672D+G753E +S754E+A769D+L775A+V800P+D801G+K875T+N892Y, Q89Y+E229S+N440K+S582K+A624E+N672D+G753E+S754E+A769D+L775A+D801G+K875T+N892Y, E229S+S635E+T649K+I656V+N672D+P752K+G753E+A769D+L775A+D801G+A843P+K875T+N892Y, E229S+S635E+T649K+I656V+N672D+G753E+S754E+S757D+A769D+L775A+D801G+K875T+N892Y, E229S+N440K+S582K+N672D+G753E+S754E+A769D+L775A+D801G+A843P+K875T+N892Y+N1008D, E229S+N440K+S582K+A624E+N672D+G753E+S754E +A769D+L775A+V800P+D801G+K875T+N892Y, A190Q+E229S+S635E+T649K+I656V+N672D+P752K+G753E+A769D+L775A+D801G+A843P+K875T+N892Y, A190Q+E229S+S582K+N672D+G753E+S754E+A769D+L775A+D801G+K875T+N892Y, E229S+N440K+S582K+N672D+P752R+G753E+S754E+S757D+A769D+L775A+D801G+K875T+N892Y +N1008D, E229S+S582K+S635E+N672D+P752R+G753E+S754E+A769D+L775A+D801G+K875T+N892Y+N1008D, A190Q+E229S+N440K+S582K+A624E+S635E+N672D +G753E+S754E+A769D+L775A+D801G+K875T+N892Y, E229S+I234V+A492L+S582K+N672D+G753E+S754E+A769D+L775A+D801G+K875T+N892Y, A190Q+E229S+K360G+D458S+S582K+T664K+N672D+G753E+S754E+A769D+L775A+D801G+K875T+N892Y +N1008D, S100D+E229S+K360G+D458S+S582K+N672D+G753E+S754E+S757D+A769D+L775A+D801G+A843P+K875T+N892Y+T915A+N1008D, E229S+N440K+S582K +A624E+S635E+N672D+G738L+G753E+S754E+S757D+A769D+L775A+D801G+K875T +N892Y, S100D+E229S+K360G+D458S+S582K+N672D+G753E+S754E+A769D+L775A +D801G+K875T+N892Y+N1008D, A190Q+E229S+D458S+T631N+N672D+G753E+S754E+A769D+L775A+D801G+A843P+K875T+N892Y, A190Q+E229S+K360G+D458S+S582K+N672D+G753E+S754E+A769D+L775A+D801G+K875T+N892Y+N1008D, E229S S635E+T649K+I656V+N672D+G753E+S754R+S757D+A769D+L775A+D801G+A843P +K875T+N892Y, E229S+D458S+S582K+T631N+S635E+N672D+M728V+G753E+S754E +S757D+A769D+L775A+D801G+K875T+N892Y, A190Q+E229S+K360G+D458S+S582K +N672D+G753E+S754E+S757D+A769D+L775A+D801G+K875T+N892Y+N1008D, E229S+A492L+S635E+T649K+I656V+N672D+G753E+S757D+A769D+L775A+D801G+K875T+N892Y, S100D+A190Q+E229S+K360G+D458S+S582K+N672D+G753E+S754E+A769D+L775A+D801G+K875T+N892Y+N1008D, A190Q+E229S+I234V+S582K+N672D+G753E+S754E+S757D+A769D+L775A+D801G+K875T+N892Y, E229S+N399K+D458S+A492H+K567R+S582K+S635E+T649K+N672D+G753E+S754E+A769D+L775A+D777R +D801G+K875T+N892Y, E229S+D458S+A492L+T631N+N672D+G753E+S754E+S757D +A769D+L775A+D801G+K875T+N892Y, E229S+D458S+A492H+K567R+S582K+S635E +N672D+G753E+S754E+A769D+L775A+D777R+D801G+K875T+N892Y, S100D+E229S +K360G+D458S+S582K+N672D+G753E+S754E+S757D+A769D+L775A+D801G+A843P+K875T+N892Y+N1008D, E229S+N399K+D458S+K567R+S582K+S635E+N672D +G753E+S754E+A769D+L775A+D777R+D801G+K875T+N892Y.

In a preferred embodiment, the detergent composition comprises a xanthan lyase variant comprising a set of alterations selected from the group consisting of E229N+N672D+P752K+G753E+A769D+L775A+D801G+K875T+N892Y, E229S+N672D+G753E+S754E+A769D+L775A+D801G+K875T+N892Y, E229S+N672D+P752R+G753E+S754E+A769D+L775A+D801G+K875T+N892Y, A190Q+E229S+I234V+A624E+N672D+G753E+S754E+A769D+L775A+D801G+K875T, A190Q+E229S+T631N+N672D+I703L+P752K+G753E+A769D+L775A+D801G+K875T, A190Q+E229S+I234V+S582K+N672D+G753E+S754E+S757D+A769D+L775A+D801G+K875T+N892Y, E229S+N440K+S582K+A624E+S635E+N672D+G738L+G753E+S754E+S757D+A769D+L775A+D801G+K875T+N892Y, and S100D+E229S+K360G+D458S+S582K+N672D+G753E+S754E+S757D+A769D+L775A+D801G+A843P +K875T+N892Y+N1008D, wherein said positions correspond to amino acid positions of SEQ ID NO:2.

In a particular aspect, the xanthan lyase variant has besides the aforementioned alterations no further alterations relative to the parent enzyme of SEQ ID NO:2, i.e. the remaining sequence is identical to SEQ ID NO:2.

### Further Preferred Embodiments

In various embodiments, the detergent composition comprises preferred endoglucanase variants in combination with the preferred xanthan lyase variants.

In a preferred embodiment, the detergent composition thus comprises

(A) an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71 E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1; and/or

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D+L775A+D801G+K875T+N892Y, and (B8) S100D+E229S+K360G+D458S+S582K+N672D+G753E+S754E+S757D+A769D+L775A+D801G+A843P+K875T+N892Y +N1008D, wherein said positions correspond to amino acid positions of SEQ ID NO:2; and

(C) an enzyme stabilizing component.

In a further preferred embodiment, the detergent composition comprises

(A) an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71 E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1; and

(B) a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D+L775A+D801G+K875T+N892Y, and (B8) S100D+E229S+K360G+D458S+S582K+N672D+G753E+S754E+S757D+A769D+L775A+D801G+A843P+K875T+N892Y +N1008D, wherein said positions correspond to amino acid positions of SEQ ID NO:2; and

(C) an enzyme stabilizing component, wherein the enzyme stabilizing component is selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and formates, more preferably being sodium formate.

In a particular preferred embodiment, the detergent composition comprises

(A) an endoglucanase variant having at least 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71 E+A186P+E408D+Y579W+I602T+A651P+A688G+V756Y, (A4) N18G+N189K +E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1; and

(B) a xanthan lyase variant having at least 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D+L775A+D801G+K875T+N892Y, and (B8) S100D+E229S+K360G+D458S+S582K+N672D+G753E+S754E+S757D+A769D+L775A+D801G+A843P +K875T+N892Y+N1008D, wherein said positions correspond to amino acid positions of SEQ ID NO:2; and

(C) an enzyme stabilizing component, wherein the enzyme stabilizing component is selected from glycerol and sodium formate, preferably being sodium formate.

In one embodiment, the endoglucanase and/or the xanthan lyase variant do not comprise any further substitution besides those explicitly mentioned above, i.e. the remainder of the sequence is identical to that of the parent enzyme as set forth in SEQ ID NO:1 and SEQ ID NO:2, respectively.

In one embodiment, the detergent composition comprises the endoglucanase variant (A1), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A2), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A3), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A4), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A5), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A6), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A7), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the endoglucanase variant (A8), as defined above, in combination with any one of the xanthan lyase variants (B1), (B2), (B3), (B4), (B5), (B6), (B7) and (B8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B1), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B2), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B3), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B4), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B5), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B6), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B7), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

In one embodiment, the detergent composition comprises the xanthan lyase variant (B8), as defined above, in combination with any one of the endoglucanase variants (A1), (A2), (A3), (A4), (A5), (A6), (A7) and (A8), as defined above, wherein the respective enzymes preferably have at least 90%, 91%, 92%, 93%, 94%, 95%, 96%, 97%, 98% or 99% sequence identity to SEQ ID NO:1 and SEQ ID NO:2, respectively, preferably are identical to their respective parent sequence with the exception of the substitutions explicitly mentioned herein.

If not explicitly indicated otherwise, all the endoglucanase and/or xanthan lyase variants, as described above, may further comprise one or more additional alterations at one or more (e.g. several) other positions in any of the regions described herein.

The amino acid changes may be of a minor nature, that is conservative amino acid substitutions or insertions that do not significantly affect the folding and/or activity of the protein, small deletions, typically of 1-30 amino acids, small amino- or carboxyl-terminal extensions, such as an amino-terminal methionine residue, a small linker peptide of up to 20-25 residues or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. The identity of essential amino acids can also be inferred from an alignment with a related polypeptide.

In one embodiment, the detergent composition additionally comprises one or more further detergent components, preferably a surfactant.

In one embodiment, the detergent composition comprises 0.000001 to 10 wt.-%, such as 0.00001 to 5 wt.-%, preferably 0.0001 to 1 wt.-%, more preferably 0.0005 to 0.5 wt.-%, even more preferably 0.001 to 0.1 wt.-%, of said endoglucanase variant (active enzyme per ml detergent composition) and/or 0.000001 to 10 wt.-%, such as 0.00001 to 5 wt.-%, preferably 0.0001 to 1 wt.-%, more preferably 0.0005 to 0.5 wt.-%, even more preferably 0.001 to 0.1 wt.-%, of said xanthan lyase variant (active enzyme per ml detergent composition) and/or 0.001 to 20 wt.-%, such as 0.005 to 15 wt.-%, preferably 0.01 to 10 wt.-%, more preferably 0.1 to 5.5 wt.-%, even more preferably 0.25 to 1.5 wt.-%, of said enzyme stabilizing agent (by weight of the detergent composition).

In one embodiment, the detergent composition comprises said endoglucanase variant and/or said xanthan lyase variant and said enzyme stabilizing agent in a weight ratio (enzyme:stabilizer) of 0.00001:1 to 0.1:2, preferably 0.0001:1 to 0.01:1, more preferably 0.0005:1 to 0.001:1.

Without being bound to the theory, it is conceivable that the addition of the enzyme stabilizing component directly interacts with the natively folded enzymes and/or influences the interaction of the enzymes and other detergent ingredients like surfactants, chelators, water, and others.

In one embodiment, the preferred endoglucanase and/or xanthan lyase variants have an improved stability in a detergent composition containing such enzyme stabilizing component(s) compared to a detergent composition not containing such enzyme stabilizing component(s).

In this respect, in a preferred embodiment, the detergent composition comprises

(A) 0.0005 to 0.5 wt.-%, preferably 0.001 to 0.1 wt.-%, of an endoglucanase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71 E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R +Y934G, wherein said positions correspond to amino acid positions of SEQ ID NO:1;

(B) 0.0005 to 0.5 wt.-%, preferably 0.001 to 0.1 wt.-%, of a xanthan lyase variant having at least 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D+L775A+D801G+K875T +N892Y, and (B8) S100D+E229S+K360G+D458S+S582K+N672D+G753E+S754E+S757D +A769D+L775A+D801G+A843P+K875T+N892Y+N1008D, wherein said positions correspond to amino acid positions of SEQ ID NO:2;

(C) 0.1 to 5.5 wt.-%, preferably 0.25 to 1.5 wt.-%, of an enzyme stabilizing component, wherein the enzyme stabilizing component is selected from the group consisting of glycerol and formates, preferably being sodium formate; and

(D) 0 to 99 wt. % of at least one further detergent component, preferably a surfactant.

In a particular preferred embodiment, the detergent composition comprises

(A) 0.0005 to 0.5 wt.-%, preferably 0.001 to 0.1 wt.-%, of an endoglucanase variant having at least 90%, 91%, 92%, 93%, 94%, 95%, 96%, 97%, 98% or 99% sequence identity to SEQ ID NO:1 and comprising a set of alterations selected from the group consisting of (A1) A559N+Y579W+T697G, (A2) K512P+A559N+Y579W+T697G, (A3) N18G+A71E+A186P+E408D+Y579W+I602T+A651P+A688G+V756Y, (A4) N18G+N189K+E408D+A559N+Y579W+A688G+T697G+V756Y+K921R+Y934G, (A5) S313D+E408D, (A6) R880K+N905D+K921R+Y934G, (A7) I302D+S313D+E408D+Y579W+I602T+A651P+T697G+R880K+K921R+Y934G, and (A8) N216Q+S313D+E408D+D476R+Y579W+I602T+F638N+A651P+T697G+W719R+R880K+T887K+K921R+Y934G, wherein said positions correspond to amino acid positions of SEQ ID NO:1;

(B) 0.0005 to 0.5 wt.-%, preferably 0.001 to 0.1 wt.-%, of a xanthan lyase variant having at least 90%, 91%, 92%, 93%, 94%, 95%, 96%, 97%, 98% or 99% sequence identity to SEQ ID NO:2 and comprising a set of alterations selected from the group consisting of (B1) E229N+N672D+P752K+G753E+A769D+L775A+D801G+K875T+N892Y, (B2) E229S+N672D+G753E+S754E+A769D+L775A+D801G+K875T+N892Y, (B3) E229S+N672D+P752R+G753E+S754E+A769D+L775A+D801G+K875T+N892Y, (B4) A190Q+E229S+I234V+A624E+N672D+G753E+S754E+A769D+L775A+D801G+K875T, (B5) A190Q+E229S+T631N+N672D+I703L+P752K+G753E+A769D+L775A+D801G+K875T, (B6) A190Q+E229S+I234V+S582K+N672D+G753E+S754E+S757D+A769D+L775A+D801G+K875T+N892Y, (B7) E229S+N440K+S582K+A624E+S635E+N672D+G738L+G753E+S754E+S757D+A769D +L775A+D801G+K875T+N892Y, and (B8) S100D+E229S+K360G+D458S+S582K+N672D +G753E+S754E+S757D+A769D+L775A+D801G+A843P+K875T+N892Y+N1008D, wherein said positions correspond to amino acid positions of SEQ ID NO:2;

(C) 0.1 to 5.5 wt.-%, preferably 0.25 to 1.5 wt.-% of an enzyme stabilizing component, wherein the enzyme stabilizing component is sodium formate; and

(D) 0 to 99 wt. % of at least one further detergent component, preferably a surfactant.

Detergent Compositions

In one certain aspect, the preferred endoglucanase variants and/or xanthan lyase variants have an improved stability in a detergent composition containing enzyme stabilizing component selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and formates, more preferably being sodium formate, compared to a detergent composition not containing such enzyme stabilizing component(s), wherein activity and/or stability in detergent is measured as disclosed in examples 2 and 3 herein.

Besides said endoglucanase variants and/or xanthan lyase variants, the detergent composition may comprise additional components. The choice of additional components is within the skill of the artisan and includes further enzymes and conventional ingredients, including the exemplary non-limiting components set forth below. The choice of components may include, for fabric care, the consideration of the type of fabric to be cleaned, the type and/or degree of soiling, the temperature at which cleaning is to take place, and the formulation of the detergent product. Although components mentioned below are categorized by general header according to a particular functionality, this is not to be construed as a limitation, as a component may comprise additional functionalities as will be appreciated by the skilled artisan.

The detergent composition may be suitable for the laundering of textiles such as e.g. fabrics, cloths or linen, or for cleaning hard surfaces such as e.g. floors, tables, or dish wash.

In one embodiment, an endoglucanase variant, as described herein, may be added to a detergent composition in an amount corresponding to 0.0001 to 200 mg, such as 0.0005 to 100 mg, preferably 0.001 to 30 mg, more preferably 0.005 to 8 mg, even more preferably 0.01 to 2 mg, of enzyme protein per liter of wash liquor.

In one embodiment, an xanthan lyase variant, as described herein, may be added to a detergent composition in an amount corresponding to 0.0001 to 200 mg, such as 0.0005 to 100 mg, preferably 0.001 to 30 mg, more preferably 0.005 to 8 mg, even more preferably 0.01 to 2 mg, of enzyme protein per liter of wash liquor.

In some embodiments, each an endoglucanase variant, as described herein, and a xanthan lyase variant, as described herein, may be added to a detergent composition each in an amount corresponding to 0.0001 to 200 mg of enzyme protein, such as 0.0005 to 100 mg, preferably 0.001 to 30 mg, more preferably 0.005 to 8 mg, even more preferably 0.01 to 2 mg, of enzyme protein per liter of wash liquor.

A composition for use in automatic dishwash (ADW) may include 0.0001 to 50 wt.-%, such as 0.001 to 20 wt.-%, preferably 0.01 to 10 wt.-%, more preferably 0.05 to 5 wt.-%, even more preferably 0.001 to 0.1 wt.-%, of each of the enzyme proteins by weight of the composition.

A composition for use in laundry granulation or a solid/granular laundry composition in general may include 0.0001 to 50 wt.-%, such as 0.001 to 20 wt.-%, preferably 0.01 to 10 wt.-%, more preferably 0.05 to 5 wt.-%, even more preferably 0.001 to 0.1 wt.-%, of each of the enzyme proteins by weight of the composition.

A composition for use in laundry liquid may include 0.0001 to 10 wt.-%, such as 0.001 to 7 wt.%, preferably 0.1 to 5 wt.-%, more preferably 0.001 to 0.1 wt.-%, of each of the enzyme proteins by weight of the composition.

A composition for use in automatic dishwash (ADW) may include 0.01 to 20 wt.-%, such as 0.1 to 10 wt.-%, preferably 0.2 to 8 wt.-%, more preferably 0.5 to 5 wt.-%, even more preferably 0.75 to 2.5 wt.-%, of said enzyme stabilizing component by weight of the composition.

A composition for use in laundry granulation or a solid/granular laundry composition in general may include 0.01 to 20 wt.-%, such as 0.1 to 10 wt.-%, preferably 0.2 to 8 wt.-%, more preferably 0.5 to 5 wt.-%, even more preferably 0.75 to 2.5 wt.-%, of said enzyme stabilizing component by weight of the composition.

A composition for use in laundry liquid may include 0.01 to 20 wt.-%, such as 0.1 to 10 wt.-%, preferably 0.2 to 8 wt.-%, more preferably 0.5 to 5 wt.-%, even more preferably 0.75 to 2.5 wt.-%, of said enzyme stabilizing component by weight of the composition.

A composition for use in automatic dishwash (ADW) may include said endoglucanase variant and/or said xanthan lyase variant and said enzyme stabilizing agent in a weight ratio (enzyme:stabilizer) of 0.00001:1 to 0.1:2, preferably 0.0001:1 to 0.01:1, more preferably 0.0005:1 to 0.001:1.

A composition for use in laundry granulation or a solid/granular laundry composition in general may include said endoglucanase variant and/or said xanthan lyase variant and said enzyme stabilizing agent in a weight ratio (enzyme:stabilizer) of 0.00001:1 to 0.1:2, preferably 0.0001:1 to 0.01:1, more preferably 0.0005:1 to 0.001:1.

A composition for use in laundry liquid may include said endoglucanase variant and/or said xanthan lyase variant and said enzyme stabilizing agent in a weight ratio (enzyme:stabilizer) of 0.00001:1 to 0.1:2, preferably 0.0001:1 to 0.01:1, more preferably 0.0005:1 to 0.001:1.

Further to the specific enzyme stabilizing component, as described herein, the enzyme(s) of the detergent composition may be stabilized using conventional stabilizing agents, e.g. a polyol such as propylene glycol or glycerol, a sugar or sugar alcohol, lactic acid, boric acid, or a boric acid derivative, e.g. an aromatic borate ester, or a phenyl boronic acid derivative such as 4-formyl phenyl boronic acid (4-FPBA), and the composition may be formulated as described in e.g. WO 92/19709 and WO 92/19708.

In certain markets different wash conditions and, as such, different types of detergents are used. This is disclosed in e.g. EP 1025240. In Asia (Japan) a low detergent concentration system is used, while the United States uses a medium detergent concentration system, and Europe uses a high detergent concentration system. A low detergent concentration system includes detergents where less than about 800 ppm of detergent components are present in the wash water. Japanese detergents are typically considered low detergent concentration system as they have approximately 667 ppm of detergent components present in the wash water. A medium detergent concentration includes detergents where between about 800 ppm and about 2000 ppm of detergent components are present in the wash water. North American detergents are generally considered to be medium detergent concentration systems as they have approximately 975 ppm of detergent components present in the wash water. A high detergent concentration system includes detergents where greater than about 2000 ppm of detergent components are present in the wash water. European detergents are generally considered to be high detergent concentration systems as they have approximately 4500 to 5000 ppm of detergent components in the wash water. Latin American detergents are generally high suds phosphate builder detergents and the range of detergents used in Latin America can fall in both the medium and high detergent concentrations as they range from 1500 to 6000 ppm of detergent components in the wash water. Such detergent compositions are all embodiments. A polypeptide may also be incorporated in the detergent formulations disclosed in WO 97/07202.

Surfactants

The detergent composition may comprise one or more surfactants, which may be anionic and/or cationic and/or non-ionic and/or semi-polar and/or zwitterionic, or a mixture thereof. In preferred embodiments, the detergent compositions comprise at least one surfactant. In a particular embodiment, the detergent composition includes a mixture of one or more nonionic surfactants and one or more anionic surfactants. The surfactant(s) is/are typically present at a level of from about 0.1 to 60 wt.-%, such as 1 to 40 wt.-%, 3 to 20 wt.-% or 3 to 10 wt.-%. The surfactant(s) is/are chosen based on the desired cleaning application, and includes any conventional surfactant(s) known in the art. Any surfactant known in the art for use in detergents may be utilized.

When included therein, the detergent will usually comprise from about 1 to 40 wt.-%, such as 5 to 30 wt.-%, 5 to 15 wt.-% or 20 to 25 wt.-%, of an anionic surfactant. Non-limiting examples of anionic surfactants include sulfates and sulfonates, in particular linear alkyl benzene sulfonates (LAS), isomers of LAS, branched alkyl benzene sulfonates (BABS), phenyl alkane sulfonates, alpha-olefin sulfonates (AOS), olefin sulfonates, alkene sulfonates, alkane-2,3-diylbis(sulfates), hydroxy alkane sulfonates and disulfonates, alkyl sulfates (AS) such as sodium dodecyl sulfate (SDS), fatty alcohol sulfates (FAS), primary alcohol sulfates (PAS), alcohol ether sulfates (AES or AEOS or FES, also known as alcohol ethoxy sulfates or fatty alcohol ether sulfates), secondary alkane sulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenyl succinic acid, dodecenyl/tetradecenyl succinic acid (DTSA), fatty acid derivatives of amino acids, diesters and monoesters of sulfo succinic acid or soap, and combinations thereof.

When included therein, the detergent will usually comprise from about 0 to 10 wt.-% of a cationic surfactant. Non-limiting examples of cationic surfactants include alkyl dimethyl ethanolamine quat (ADMEAQ), cetyl trimethyl ammonium bromide (CTAB), dimethyl distearyl ammonium chloride (DSDMAC), and alkyl benzyl dimethyl ammonium, alkyl quaternary ammonium compounds, alkoxylated quaternary ammonium (AQA) compounds, and combinations thereof.

When included therein, the detergent will usually comprise from about 0.2 to 40 wt.-% of a non-ionic surfactant, e.g. 0.5 to 30 wt.-%, in particular 1 to 20 wt.-%, 3 to 10 wt.-%, 3 to 5 wt.-% or 8 to 12 wt.-%. Non-limiting examples of non-ionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkyl phenol ethoxylates (APE), nonyl phenol ethoxylates (NPE), alkyl polyglycosides (APG), alkoxylated amines, fatty acid monoethanolamides (FAM), fatty acid diethanol amides (FADA), ethoxylated fatty acid monoethanolamides (EFAM), propoxylated fatty acid monoethanolamides (PFAM), polyhydroxy alkyl fatty acid amides, or N-acyl N-alkyl derivatives of glucosamine (glucamides, GA, or fatty acid glucamide, FAGA), as well as products available under the trade names SPAN and TWEEN, and combinations thereof.

When included therein, the detergent will usually comprise from about 0 to 10 wt.-% of a semi-polar surfactant. Non-limiting examples of semipolar surfactants include amine oxides (AO) such as alkyl dimethyl amine oxide, N-(coco alkyl)-N,N-dimethyl amine oxide and N-(tallow-alkyl)-N,N-bis-(2-hydroxy ethyl) amine oxide, fatty acid alkanol amides and ethoxylated fatty acid alkanol amides, and combinations thereof.

When included therein, the detergent will usually comprise from about 0 to 10 wt.-% of a zwitterionic surfactant. Non-limiting examples of zwitterionic surfactants include betaine, alkyl dimethyl betaine, sulfo betaine, and combinations thereof.

Hydrotropes

A hydrotrope is a compound that solubilizes hydrophobic compounds in aqueous solutions (or oppositely, polar substances in a non-polar environment). Typically, hydrotropes have both hydrophilic and a hydrophobic character (so-called amphiphilic properties as known from surfactants); however, the molecular structure of hydrotropes generally do not favor spontaneous self-aggregation. Hydrotropes do not display a critical concentration above which self-aggregation occurs as found for surfactants and lipids forming micellar, lamellar or other well defined meso-phases. Instead, many hydrotropes show a continuous-type aggregation process where the sizes of aggregates grow as concentration increases. However, many hydrotropes alter the phase behavior, stability, and colloidal properties of systems containing substances of polar and non-polar character, including mixtures of water, oil, surfactants, and polymers. Hydrotropes are classically used across industries from pharma, personal care, food, to technical applications. Use of hydrotropes in detergent compositions allow more concentrated formulations of surfactants (as in the process of compacting liquid detergents by removing water) without inducing undesired phenomena such as phase separation or high viscosity.

The detergent may comprise 0 to 5 wt.-%, such as 0.5 to 5 wt.-% or 3 to 5 wt.-%, of a hydrotrope. Any hydrotrope known in the art for use in detergents may be utilized. Non-limiting examples of hydrotropes include sodium benzene sulfonate, sodium p-toluene sulfonate (STS), sodium xylene sulfonate (SXS), sodium cumene sulfonate (SCS), sodium cymene sulfonate, amine oxides, alcohols and polyglycol ethers, sodium hydroxy naphthoate, sodium hydroxy naphthalene sulfonate, sodium ethyl hexyl sulfate, and combinations thereof.

Builders and Cobuilders

The detergent composition may comprise about 0 to 65 wt.-%, such as 5 to 45 wt.-%, of a detergent builder or cobuilder, or a mixture thereof. In a dish wash detergent, the level of builder is typically 40 to 65 wt.-%, particularly 50 to 65 wt.-%. The builder and/or cobuilder may particularly be a chelating agent that forms water-soluble complexes with Ca²⁺ and Mg²⁺. Any builder and/or cobuilder known in the art for use in laundry detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g. SKS-6 from Hoechst), ethanol amines such as 2-amino ethan-1-ol (MEA), diethanolamine (DEA, also known as imino diethanol), triethanolamine (TEA, also known as 2,2′,2″-nitrilo triethanol), and carboxy methyl inulin (CMI), and combinations thereof.

The detergent composition may also comprise 0 to 20 wt.-%, such as 5 to 10 wt.-%, of a detergent cobuilder, or a mixture thereof. The detergent composition may include a cobuilder alone, or in combination with a builder, e.g. a zeolite builder. Non-limiting examples of cobuilders include homopolymers of polyacrylates or copolymers thereof, such as poly (acrylic acid) (PAA) or copoly (acrylic acid/maleic acid) (PAA/PMA). Further non-limiting examples include citrate, chelators such as amino carboxylates, amino polycarboxylates and phosphonates, and alkyl- or alkenyl succinic acid. Additional specific examples include 2,2′,2″-nitrilo triacetic acid (NTA), ethylene diamine tetraacetic acid (EDTA), diethylene triamine pentaacetic acid (DTPA), imino disuccinic acid (IDS), ethylene diamine-N,N′-disuccinic acid (EDDS), methyl glycine diacetic acid (MGDA), glutamic acid-N,N-diacetic acid (GLDA), 1-hydroxy ethane-1,1-diphosphonic acid (HEDP), ethylene diamine tetra-(methylene phosphonic acid) (EDTMPA), diethylene triamine pentakis (methylene phosphonic acid) (DTPMPA or DTMPA), N-(2-hydroxy ethyl) imino diacetic acid (EDG), aspartic acid-N-monoacetic acid (ASMA), aspartic acid-N,N-diacetic acid (ASDA), aspartic acid-N-monopropionic acid (ASMP), imino disuccinic acid (IDA), N-(2-sulfo methyl) aspartic acid (SMAS), N-(2-sulfo ethyl) aspartic acid (SEAS), N-(2-sulfo methyl) glutamic acid (SMGL), N-(2-sulfo ethyl) glutamic acid (SEGL), N-methyl imino diacetic acid (MIDA), α-alanine-N,N-diacetic acid (α-ALDA), serine-N,N-diacetic acid (SEDA), isoserine-N,N-diacetic acid (ISDA), phenyl alanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfo methyl-N,N-diacetic acid (SMDA), N-(2-hydroxy ethyl) ethylidene diamine-N,N′,N′-triacetate (HEDTA), diethanol glycine (DEG), diethylene triamine penta (methylene phosphonic acid) (DTPMP), amino tris (methylene phosphonic acid) (ATMP), and combinations and salts thereof. Further exemplary builders and/or cobuilders are described in e.g. WO 2009/102854, U.S. Pat. No. 5,977,053.

Bleaching Systems

The detergent may comprise 0 to 50 wt.-%, such as 0.1 to 25 wt.-%, of a bleaching system. Any bleaching system known in the art for use in laundry detergents may be utilized. Suitable bleaching system components include bleaching catalysts, photobleaches, bleach activators, sources of hydrogen peroxide such as sodium percarbonate and sodium perborates, preformed peracids and mixtures thereof.

Suitable preformed peracids include, but are not limited to, peroxy carboxylic acids and salts, percarbonic acids and salts, perimidic acids and salts, peroxy monosulfuric acids and salts, e.g. Ox-one®, and mixtures thereof.

Non-limiting examples of bleaching systems include peroxide based bleaching systems, which may comprise, e.g., an inorganic salt, including alkali metal salts such as sodium salts of perborate (usually mono- or tetra-hydrate), percarbonate, persulfate, perphosphate, persilicate salts, in combination with a peracid-forming bleach activator.

The term bleach activator is meant herein as a compound which reacts with peroxygen bleach like hydrogen peroxide to form a peracid. The peracid thus formed constitutes the activated bleach. Suitable bleach activators to be used herein include those belonging to the class of esters amides, imides or anhydrides. Suitable examples are tetra cetyl ethylene diamine (TAED), sodium 4-[(3,5,5-trimethyl hexanoyl)oxy] benzene sulfonate (ISONOBS), diperoxy dodecanoic acid, 4-(dodecanoyl oxy) benzene sulfonate (LOBS), 4-(decanoyl oxy) benzene sulfonate, 4-(decanoyl oxy) benzoate (DOBS), 4-(nonanoyl oxy) benzene sulfonate (NOBS), and/or those disclosed in WO 98/17767. A particular family of bleach activators of interest was disclosed in EP 0624154 and particularly preferred in that family is acetyl triethyl citrate (ATC). ATC or a short chain triglyceride like triacetin has the advantage that it is environmentally friendly as it eventually degrades into citric acid and alcohol. Furthermore, acetyl triethyl citrate and triacetin has a good hydrolytic stability in the product upon storage and it is an efficient bleach activator. Finally, ATC provides a good building capacity to the laundry additive.

Alternatively, the bleaching system may comprise peroxy acids of, e.g., the amide, imide, or sulfone type. The bleaching system may also comprise peracids such as 6-(phthalo imido) peroxy hexanoic acid (PAP).

The bleaching system may also include a bleach catalyst. In some embodiments the bleach component may be an organic catalyst selected from the group consisting of organic catalysts having the following formulae:

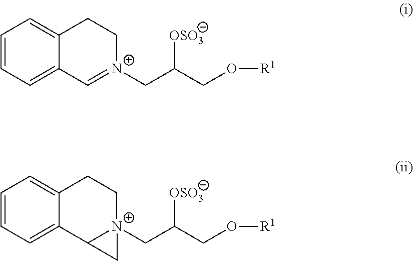

(iii) and mixtures thereof;

wherein each R¹ is independently a branched alkyl group containing from 9 to 24 carbons or linear alkyl group containing from 11 to 24 carbons, preferably each R¹ is independently a branched alkyl group containing from 9 to 18 carbons or linear alkyl group containing from 11 to 18 carbons, more preferably each R¹ is independently selected from the group consisting of 2-propyl heptyl, 2-butyl octyl, 2-pentyl nonyl, 2-hexyl decyl, n-dodecyl, n-tetradecyl, n-hexadecyl, n-octadecyl, isononyl, isodecyl, isotridecyl and isopentadecyl.

Other exemplary bleaching systems are described in e.g. WO 2007/087258, WO 2007/087244, WO 2007/087259 and WO 2007/087242.

Suitable photobleaches may be e.g. sulfonated zinc phthalo cyanine.

Polymers

The detergent may comprise 0 to 10 wt.-%, such as 0.5 to 5 wt.-%, 2 to 5 wt.-%, 0.5 to 2 wt.-% or 0.2 or 1 wt.-%, of a polymer. Any polymer known in the art for use in detergents may be utilized. The polymer may function as a cobuilder as mentioned above, or may provide anti-redeposition, fiber protection, soil release, dye transfer inhibition, grease cleaning and/or anti-foaming properties. Some polymers may have more than one of the above-mentioned properties and/or more than one of the below-mentioned motifs. Exemplary polymers include carboxy methyl cellulose (CMC), poly (vinyl alcohol) (PVA), poly (vinyl pyrrolidone) (PVP), poly (ethylene glycol) or poly (ethylene oxide) (PEG), ethoxylated poly (ethylene imine), carboxy methyl inulin (CMI), and polycarboxylates such as PAA, PAA/PMA, polyaspartic acid, and lauryl methacrylate/acrylic acid copolymers, hydrophobically modified CMC (HM-CMC) and silicones, copolymers of terephthalic acid and oligomeric glycols, copolymers of poly (ethylene terephthalate) and poly (oxyethene terephthalate) (PET-POET), PVP, poly (vinyl imidazole) (PVI), poly (vinyl pyridine-N-oxide) (PVPO or PVPNO) and polyvinyl pyrrolidone vinyl imidazole (PVPVI). Further exemplary polymers include sulfonated polycarboxylates, polyethylene oxide and polypropylene oxide (PEO-PPO) and diquaternium ethoxy sulfate. Other exemplary polymers are disclosed in, e.g. WO 2006/130575. Salts of the abovementioned polymers are also contemplated.

Fabric Hueing Agent

The detergent compositions may also comprise fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Color Index (C.I.) classifications of Direct Blue, Direct Red, Direct Violet, Acid Blue, Acid Red, Acid Violet, Basic Blue, Basic Violet and Basic Red, or mixtures thereof, as described in e.g. WO 2005/003274, WO 2005/003275, WO 2005/003276 and EP 1876226.

The detergent composition preferably comprises from about 0.00003 to 0.2 wt.-%, such as 0.00008 to 0.05 wt.-% or 0.0001 to 0.04 wt.-%, of a fabric hueing agent. The composition may comprise from 0.0001 to 0.2 wt.-% fabric hueing agent, this may be especially preferred when the composition is in the form of a unit dose pouch. Suitable hueing agents are also disclosed in e.g. WO 2007/087257 and WO 2007/087243.

Additional Enzymes

The detergent composition may comprise one or more additional enzymes such as proteases, amylases, lipases, cutinases, cellulases, endoglucanases, xyloglucanases, pectinases, pectin lyases, xanthanases, peroxidases, haloperoxygenases, oxidoreductases, catalases and mannanases, or any mixture thereof.

In general, the properties of the selected enzyme(s) should be compatible with the selected detergent, (i.e. pH-optimum, compatibility with other enzymatic and non-enzymatic ingredients, etc.), and the enzyme(s) should be present in effective amounts.

Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, or the fungal cellulases produced from Humicola insolens, Myceliophthora thermophila and Fusarium oxysporum, disclosed in U.S. Pat. Nos. 4,435,307, 5,648,263, 5,691,178, 5,776,757 and WO 89/09259.

Especially suitable cellulases are the alkaline or neutral cellulases having color care benefits. Examples of such cellulases are described in EP 0495257, EP 0531372, WO 96/11262, WO 96/29397, WO 98/08940. Other examples are cellulase variants are described in WO 94/07998, EP 0531315, U.S. Pat. Nos. 5,457,046, 5,686,593, 5,763,254, WO 95/24471, WO 98/12307 and WO 99/01544.

Examples of cellulases exhibiting endo-β-1,4-glucanase activity (EC 3.2.1.4) are described in WO 2002/099091.

Other examples of cellulases include the GH45 cellulases described in WO 96/29397, and especially variants thereof having substitution, insertion and/or deletion at one or more of the positions corresponding to the following positions in SEQ ID NO:8 of WO 2002/099091: 2, 4, 7, 8, 10, 13, 15, 19, 20, 21, 25, 26, 29, 32, 33, 34, 35, 37, 40, 42, 42a, 43, 44, 48, 53, 54, 55, 58, 59, 63, 64, 65, 66, 67, 70, 72, 76, 79, 80, 82, 84, 86, 88, 90, 91, 93, 95, 95d, 95h, 95j, 97, 100, 101, 102, 103, 113, 114, 117, 119, 121, 133, 136, 137, 138, 139, 140a, 141, 143a, 145, 146, 147, 150e, 150j, 151, 152, 153, 154, 155, 156, 157, 158, 159, 160c, 160e, 160k, 161, 162, 164, 165, 168, 170, 171, 172, 173, 175, 176, 178, 181, 183, 184, 185, 186, 188, 191, 192, 195, 196, 200, and/or 20, preferably selected among P19A, G20K, Q44K, N48E, Q119H or Q146R.

Commercially available cellulases include Celluzyme™, and Carezyme™ (Novozymes A/S), Clazinase™, and Puradax HA™ (Genencor International Inc./DuPont), and KAC-500(B)™ (Kao Corporation).

Proteases

The protease may be of animal, vegetable or microbial origin, including chemically or genetically modified mutants. Microbial origin is preferred. It may be an alkaline protease, such as a serine protease or a metalloprotease. A serine protease may be of the S1 family, such as trypsin, or the S8 family such as subtilisin. A metalloproteases protease may be a thermolysin from e.g. family M4, M5, M7 or M8.

The term “subtilases” refers to a subgroup of serine protease. Serine proteases are a subgroup of proteases characterized by having a serine in the active site, which forms a covalent adduct with the substrate. The subtilases may be divided into 6 subdivisions, i.e. the Subtilisin family, the Thermitase family, the Proteinase K family, the Lantibiotic peptidase family, the Kexin family and the Pyrolysin family. In one aspect, the protease may be a subtilase, such as a subtilisin or a variant hereof. Further the subtilases (and the serine proteases) are characterized by having two active site amino acid residues apart from the serine, namely a histidine and an aspartic acid residue.

Examples of subtilisins are those derived from Bacillus such as subtilisin lentus, Bacillus lentus, subtilisin Novo, subtilisin Carlsberg, Bacillus licheniformis, subtilisin BPN′, subtilisin 309, subtilisin 147 and subtilisin 168, described in WO 89/06279, and protease PD138 (WO 93/18140). Further serine protease examples are described in WO 98/20115, WO 2001/044452, WO 2001/058275, WO 2001/058276, WO 2003/006602 and WO 2004/099401.

An example of a subtilase variants may be those having mutations in any of the positions: 3, 4, 9, 15, 27, 36, 68, 76, 87, 95, 96, 97, 98, 99, 100, 101, 102, 103, 104, 106, 118, 120, 123, 128, 129, 130, 160, 167, 170, 194, 195, 199, 205, 217, 218, 222, 232, 235, 236, 245, 248, 252 and 274 (using the BPN′ numbering). More preferred the subtilase variants may comprise one of the mutations selected from the group consisting of S3T, V4I, S9R, A15T, K27R, *36D, V68A, N76D, N87S,R, *97E, A98S, S99G,D,A, S99AD, S101G,M,R, S103A, V104I, Y,N, S106A, G118V,R, H120D,N, N123S, S128L, P129Q, S130A, G160D, Y167A, R170S, A194P, G195E, V199M, V205I, L217D, N218D, M222S, A232V, K235L, Q236H, Q245R, N252K and T274A (using BPN′ numbering).

A further preferred protease is the alkaline protease from Bacillus lentus DSM 5483, as described in e.g. WO 95/23221, and variants thereof which are described in e.g. WO 92/21760, WO 95/23221, EP 1921147 and EP 1921148.

Examples of trypsin-like proteases are trypsin (e.g. of porcine or bovine origin) and the Fusarium l protease, described in WO 89/06270 and WO 94/25583. Examples of useful proteases are the variants described in WO 92/19729, WO 98/20115, WO 98/20116 and WO 98/34946, especially the variants with substitutions in one or more of the following positions: 27, 36, 57, 76, 87, 97, 101, 104, 120, 123, 167, 170, 194, 206, 218, 222, 224, 235, and 274.

Examples of metalloproteases are the neutral metalloprotease as described in WO 2007/044993.

Preferred commercially available protease enzymes include Alcalase™, Blaze Evity™, Coronase™, Duralase™, Durazym™, Esperase™, Everlase™, Kannase™, Liquanase™, Liquanase Ultra™, Ovozyme™, Polarzyme™, Primase™, Relase™, Savinase™ and Savinase Ultra™ (Novozymes A/S), Axapem™ (Gist-Brocases N.V.), BLAP and variants thereof (Henkel AG & Co. KGaA), Excellase™, FN2™, FN3™, FN4™, Maxaca™, Maxapem™, Maxatase™, Properase™, Purafast™, Purafect™, Purafect OxP™, Purafect Prime™ and Puramax™ (Genencor int./DuPont).

Lipases and Cutinases

Suitable lipases and cutinases include those of bacterial or fungal origin. Chemically modified or protein engineered mutant enzymes are included. Examples include lipase from Thermomyces, e.g. from T. lanuginosus (previously named Humicola lanuginosa) as described in EP 0258068 and EP 0305216, cutinase from Humicola, e.g. H. insolens (WO 96/13580), lipase from strains of Pseudomonas (some of these now renamed to Burkholderia), e.g. P. alcaligenes or P. pseudoalcaligenes (EP 0218272), P. cepacia (EP 0331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Further examples are lipases sometimes referred to as acyltransferases or perhydrolases, e.g. acyltransferases with homology to Candida antarctica lipase A (WO 2010/111143), acyltransferase from Mycobacterium smegmatis (WO 2005/056782), perhydrolases from the CE 7 family (WO 2009/067279), and variants of the M. smegmatis perhydrolase, in particular the S54V variant used in the commercial product Gentle Power Bleach from Huntsman Textile Effects Pte Ltd (WO 2010/100028). Other examples are lipase variants such as those described in EP 0407225, WO 92/05249, WO 94/01541, WO 94/25578, WO 95/14783, WO 95/30744, WO 95/35381, WO 95/22615, WO 96/00292, WO 97/04079, WO 97/07202, WO 2000/034450, WO 2000/060063, WO 2001/092502, WO 2007/087508 and WO 2009/109500.

Preferred commercial lipase products include Lipolase™, Lipex™, Lipolex™ and Lipoclean™ (Novozymes A/S), Lumafast (Genencor/DuPont) and Lipomax (Gist-Brocades).

Amylases

The amylase may be an α-amylase, a β-amylase or a glucoamylase and may be of bacterial or fungal origin. Chemically modified or protein engineered mutants are included. Amylases include, e.g., α-amylases obtained from Bacillus, e.g. Bacillus licheniformis (GB 1296839).

Examples of amylases are those having SEQ ID NO:3 in WO 95/10603 or variants having 90% sequence identity to SEQ ID NO:3 thereof, preferred variants being described in WO 94/02597, WO 94/18314, WO 97/43424; and SEQ ID NO:4 of WO 99/019467, as well as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444 of SEQ ID NO:3 in WO 95/10603.

Further amylases which can be used are amylases having SEQ ID NO:6 in WO 2002/010355 or variants thereof having 90% sequence identity to SEQ ID NO:6. Preferred variants of SEQ ID NO:6 are those having a deletion in positions 181 and 182 and a substitution in position 193.

Other amylase examples are hybrid α-amylases comprising residues 1 to 33 of the α-amylase derived from B. amyloliquefaciens shown in SEQ ID NO:6 of WO 2006/066594 and residues 36 to 483 of the B. licheniformis α-amylase shown in SEQ ID NO:4 of WO 2006/066594 or variants having 90% sequence identity thereof. Preferred variants of this hybrid α-amylase are those having a substitution, a deletion or an insertion in one of more of the following positions: G48, T49, G107, H156, A181, N190, M197, I201, A209 and Q264. Most preferred variants of the hybrid α-amylase comprising residues 1 to 33 of SEQ ID NO:6 of WO 2006/066594 and residues 36 to 483 of SEQ ID NO:4 of WO 2006/066594 of WO 2006/066594 are those having the substitutions: M197T, H156Y+A181T+N190F+A209V+Q264S or G48+T49+G107+H156+A18I+N190+120I+A209+Q264.

Further amylase examples are those having SEQ ID NO:6 in WO 99/19467 or variants thereof having 90% sequence identity to SEQ ID NO:6. Preferred variants of SEQ ID NO:6 are those having a substitution, a deletion or an insertion in one or more of the following positions: R181, G182, H183, G184, N195, I206, E212, E216 and K269. Particularly preferred amylases are those having deletion in positions G182+H183 or H183+G184.

Additional amylases are those having SEQ ID NO:1, SEQ ID NO:2 or SEQ ID NO:7 of WO 96/23873 or variants thereof having 90% sequence identity to SEQ ID NO:1,2 or 7. Preferred variants of SEQ ID NO:1, SEQ ID NO:2 or SEQ ID NO:7 are those having a substitution, a deletion or an insertion in one or more of the following positions: 140, 181, 182, 183, 184, 195, 206, 212, 243, 260, 269, 304 and 476. More preferred variants are those having a deletion in positions 182+183 or 183+184. Most preferred amylase variants of SEQ ID NO:1, SEQ ID NO:2 or SEQ ID NO:7 are those having a deletion in positions 183+184 and a substitution in positions 140, 195, 206, 243, 260, 304 and 476.

Other amylases which can be used are those having SEQ ID NO:2 of WO 2008/153815, SEQ ID NO:10 in WO 2001/066712 or variants thereof having 90% sequence identity to SEQ ID NO:2 of WO 2008/153815 or 90% sequence identity to SEQ ID NO:10 in WO 2001/066712. Preferred variants of SEQ ID NO:10 in WO 2001/066712 are those having a substitution, a deletion or an insertion in one of more of the following positions: 176, 177, 178, 179, 190, 201, 207, 211 and 264.

Further amylases which can be used are amylases having SEQ ID NO:2 of WO 2009/061380 or variants thereof having 90% sequence identity to SEQ ID NO:2. Preferred variants of SEQ ID NO:2 are those having a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO:2 are those having the substitution in one of more of the following positions: Q87E,R, Q98R, S125A, N128C, T131I, T165I, K178L, T182G, M201L, F202Y, N225E,R, N272E,R, S243Q,A,E,D, Y305R, R309A, Q320R, Q359E, K444E and G475K and/or deletion in position R180 and/or S181. Most preferred amylase variants of SEQ ID NO:2 are those having the substitutions: N128C+K178L +T182G+Y305R+G475K, N128C+K178L+T182G+F202Y+Y305R+D319T+G475K, S125A +N128C+K178L+T182G+Y305R+G475K or S125A+N128C+T131I+T165I+K178L+T182G +Y305R+G475K, wherein the variant optionally further comprises a substitution at position 243 and/or a deletion at position 180 and/or 181.

Other amylases are variants of SEQ ID NO:1 of WO 2016/203064 having at least 75% sequence identity to SEQ ID NO:1 thereof. Preferred variants are variants comprising a modification in one or more positions corresponding to positions 1, 54, 56, 72, 109, 113, 116, 134, 140, 159, 167, 169, 172, 173, 174, 181, 182, 183, 184, 189, 194, 195, 206, 255, 260, 262, 265, 284, 289, 304, 305, 347, 391, 395, 439, 469, 444, 473, 476, or 477 of SEQ ID NO:1, wherein said α-amylase variant has a sequence identity of at least 75% but less than 100% to SEQ ID NO:1.

Other examples of amylases are the α-amylase having SEQ ID NO:12 in WO 2001/066712 or a variant having at least 90% sequence identity to SEQ ID NO:12. Preferred amylase variants are those having a substitution, a deletion or an insertion in one of more of the following positions of SEQ ID NO:12 in WO 2001/066712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Commercially available amylases are Amplify 12L™, Amplify Prime 100L™, Duramyl™, Termamyl™, Fungamyl™, Stainzyme™, Stainzyme Plus™, Natalase™ and BAN™ (Novozymes A/S), Preferenz S210™, Excellenz S3300™, Rapidase™ and Purastar™ (Genencor/DuPont).

Peroxidases/Oxidases

Suitable peroxidases/oxidases include those of plant, bacterial or fungal origin. Chemically modified or protein engineered mutants are included. Examples of useful peroxidases include peroxidases from Coprinus, e.g. C. cinereus, and variants thereof as those described in WO 93/24618, WO 95/10602 and WO 98/15257. Commercially available peroxidases include Guardzyme™ (Novozymes A/S).

The detergent enzyme(s) may be included in a detergent composition by adding separate additives containing one or more enzymes, or by adding a combined additive comprising all of these enzymes. A detergent additive, i.e. a separate additive or a combined additive, can be formulated, e.g., as a granulate, liquid, slurry, etc. Preferred detergent additive formulations are granulates, in particular non-dusting granulates, liquids, in particular stabilized liquids, or slurries.

Non-dusting granulates may be produced, e.g. as disclosed in U.S. Pat. Nos. 4,106,991 and 4,661,452, and may optionally be coated by methods known in the art. Examples of waxy coating materials are poly-(ethylene oxide) products (polyethylene glycol, PEG) with mean molar weights of 1000 to 20000; ethoxylated nonyl phenols having from 16 to 50 ethylene oxide units; ethoxylated fatty alcohols in which the alcohol contains from 12 to 20 carbon atoms and in which there are 15 to 80 ethylene oxide units; fatty alcohols; fatty acids; and mono- and di- and triglycerides of fatty acids. Examples of film-forming coating materials suitable for application by fluid bed techniques are given in GB 1483591.

Liquid enzyme preparations may, e.g., be stabilized by adding a polyol such as propylene glycol, a sugar or sugar alcohol, lactic acid or boric acid according to established methods. Protected enzymes may be prepared according to the method disclosed in EP 0238216.

Adjunct Materials

Any detergent components known in the art for use in laundry detergents may also be utilized. Other optional detergent components include anti-corrosion agents, anti-shrink agents, anti-soil redeposition agents, anti-wrinkling agents, bactericides, binders, corrosion inhibitors, disintegrants/disintegration agents, dyes, enzyme stabilizers (including boric acid, borates, CMC and/or polyols such as propylene glycol), fabric conditioners including clays, fillers/processing aids, fluorescent whitening agents/optical brighteners, foam boosters, foam (suds) regulators, perfumes, soil-suspending agents, softeners, suds suppressors, tarnish inhibitors, and wicking agents, either alone or in combination. Any ingredient known in the art for use in laundry detergents may be utilized. The choice of such ingredients is well within the skill of the artisan.

Dispersants: The detergent compositions can also contain dispersants. In particular, powdered detergents may comprise dispersants. Suitable water-soluble organic materials include the homo- or copolymeric acids or their salts, in which the polycarboxylic acid comprises at least two carboxyl radicals separated from each other by not more than two carbon atoms. Suitable dispersants are described in e.g. Powdered Detergents, Surfactant science series volume 71, Marcel Dekker, Inc.

Dye Transfer Inhibiting Agents: The detergent compositions may also include one or more dye transfer inhibiting agents. Suitable polymeric dye transfer inhibiting agents include, but are not limited to, polyvinyl pyrrolidone polymers, polyamine-N-oxide polymers, copolymers of N-vinyl pyrrolidone and N-vinyl imidazole, polyvinyl oxazolidones and polyvinyl imidazoles or mixtures thereof. When present in a detergent composition, the dye transfer inhibiting agents may be present at levels from about 0.0001 to 10 wt.-%, such as 0.01 to 5 wt.-% or 0.1 to 3 wt.-%.

Fluorescent whitening agent: The detergent compositions may also contain additional components that may tint articles being cleaned, such as fluorescent whitening agent or optical brighteners. Where present the brightener is preferably at a level of about 0.01 to 0.5 wt.-%. Any fluorescent whitening agent suitable for use in a laundry detergent composition may be used in the detergent composition. The most commonly used fluorescent whitening agents are those belonging to the classes of diamino stilbene sulphonic acid derivatives, diary! pyrazoline derivatives and bisphenyl distyryl derivatives. Examples of diamino stilbene sulphonic acid derivative type of fluorescent whitening agents include sodium salts of 4,4′-bis-(2-diethanol amino-4-anilino-s-triazin-6-yl amino) stilbene-2,2′-disulphonate, 4,4′-bis-(2,4-dianilino-s-triazin-6-ylamino) stilbene-2,2′-disulphonate, 4,4′-bis-(2-anilino-4-(N-methyl-N-2-hydroxy ethyl amino)-s-triazin-6-yl amino) stilbene-2,2′-disulphonate, 4,4′-bis-(4-phenyl-2,1,3-triazol-2-yl) stilbene-2,2′-disulphonate, 4,4′-bis-(2-anilino-4-(1-methyl-2-hydroxy ethyl amino)-s-triazin-6-yl amino) stilbene-2,2′-disulphonate and 2-(stilbyl-4″naptho-1,2′,4,5)-1,2,3-trizole-2″-sulphonate. Preferred fluorescent whitening agents are Tinopal DMS and Tinopal CBS (Ciba-Geigy AG). Tinopal DMS is the disodium salt of 4,4′-bis-(2-morpholino-4 anilino-s-triazin-6-yl amino) stilbene disulphonate. Tinopal CBS is the disodium salt of 2,2′-bis-(phenyl styryl) disulphonate. Also preferred are fluorescent whitening agents is the commercially available Parawhite KX (Paramount Minerals and Chemicals). Other fluorescers include the 1-3-diaryl pyrazolines and the 7-alkyl amino coumarins. Suitable fluorescent brightener levels include lower levels of from about 0.01 wt.-%, such as 0.05 wt.-%, 0.1 wt.-% or 0.2 wt.-%, to upper levels of 0.5 wt.-% or 0.75 wt.-%.

Soil release polymers: The detergent compositions may also include one or more soil release polymers which aid the removal of soils from fabrics such as cotton and polyester based fabrics, in particular the removal of hydrophobic soils from polyester based fabrics. The soil release polymers may be nonionic or anionic terephthalate-based polymers, polyvinyl caprolactam and related copolymers, vinyl graft copolymers, polyester polyamides (see e.g. Chapter 7 in Powdered Detergents, Surfactant science series volume 71, Marcel Dekker, Inc). Another type of soil release polymer is amphiphilic alkoxylated grease cleaning polymers comprising a core structure and a plurality of alkoxylate groups attached to that core structure. The core structure may comprise a polyalkylene imine structure or a polyalkanol amine structure as described in detail in WO 2009/087523. Furthermore, random graft copolymers are suitable soil release polymers Suitable graft copolymers are described in more detail in WO 2007/138054, WO 2006/108856 and WO 2006/113314. Other soil release polymers are substituted polysaccharide structures especially substituted cellulosic structures such as modified cellulose derivatives such as those described in EP 1867808 or WO 2003/040279. Suitable cellulosic polymers include cellulose, cellulose ethers, cellulose esters, cellulose amides and mixtures thereof. Suitable cellulosic polymers include anionically modified cellulose, non-ionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

Anti-redeposition agents: The detergent compositions may also include one or more anti-redeposition agents such as carboxy methyl cellulose (CMC), polyvinyl alcohol (PVA), polyvinyl pyrrolidone (PVP), polyoxyethylene and/or polyethylene glycol (PEG), homopolymers of acrylic acid, copolymers of acrylic acid and maleic acid, and ethoxylated polyethylene imines. The cellulose based polymers described under soil release polymers above may also function as anti-redeposition agents.

Other suitable adjunct materials include, but are not limited to, anti-shrink agents, anti-wrinkling agents, bactericides, binders, carriers, dyes, enzyme stabilizers, fabric softeners, fillers, foam regulators, hydrotropes, perfumes, pigments, suds suppressors, solvents and structurants for liquid detergents and/or structure elasticizing agents.

Formulation of Detergent Products

The detergent composition may be in any convenient form, e.g. a bar, a homogenous tablet, a tablet having two or more layers, a pouch having one or more compartments, a regular or compact powder, a granule, a paste, a gel, or a regular, compact or concentrated liquid. There are a number of detergent formulation forms such as layers (same or different phases), pouches, as well as forms for machine dosing unit.

Pouches can be configured as single or multicompartment. It can be of any form, shape and material which is suitable for hold the composition, e.g. without allowing the release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected from polyacrylates, water soluble acrylate copolymers, methyl cellulose, carboxy methyl cellulose, sodium dextrin, ethyl cellulose, hydroxy ethyl cellulose, hydroxy propyl methyl cellulose, malto dextrin, polymethacrylates, most preferably polyvinyl alcohol copolymers and, hydroxy propyl methyl cellulose (HPMC). Preferably the level of polymer in the film is at least about 60%. Preferred average molecular weight will typically be about 20000 to about 150000. Films can also be of blend compositions comprising hydrolytically degradable and water-soluble polymer blends such as polylactide and polyvinyl alcohol (known under the Trade reference M8630 as sold by Chris Craft In. Prod. Of Gary, Ind., US) plus plasticizers like glycerol, ethylene glycerol, propylene glycol, sorbitol and mixtures thereof. The pouches can comprise a solid laundry cleaning composition or part components and/or a liquid cleaning composition or part components separated by the water-soluble film. The compartment for liquid components can be different in composition than compartments containing solids (US 2009/011970).

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20 wt.-% and up to 95 wt.-%, such as up to 70 wt.-%, 65 wt.-%, 55 wt.-%, 45 wt.-% or 35 wt.-%, water. Other types of liquids, including without limitation, alkanols, amines, diols, ethers and polyols may be included in an aqueous liquid or gel. An aqueous liquid or gel detergent may contain from 0 to 30 wt.-% organic solvent. A liquid or gel detergent may be non-aqueous.

The enzymes may be added to laundry soap bars and used for hand washing laundry, fabrics and/or textiles. The term laundry soap bar includes laundry bars, soap bars, combo bars, syndet bars and detergent bars. The types of bar usually differ in the type of surfactant they contain, and the term laundry soap bar includes those containing soaps from fatty acids and/or synthetic soaps. The laundry soap bar has a physical form which is solid and not a liquid, gel or a powder at room temperature. The term solid is defined as a physical form which does not significantly change over time, i.e. if a solid object (e.g. laundry soap bar) is placed inside a container, the solid object does not change to fill the container it is placed in. The bar is a solid typically in bar form but can be in other solid shapes such as round or oval.

Method of Producing the Composition

The present disclosure also relates to methods of producing the composition. The method may be relevant for the (storage) stability of the detergent composition, e.g. soap bar premix method (WO 2009/155557).

Uses

The present disclosure is also directed to methods for using the detergent compositions thereof, such as in any detergent application which requires the degradation of xanthan gum.

Use to Degrade Xanthan Gum

Xanthan gum has been used as an ingredient in many consumer products including foods and cosmetics and has found use in the oil industry. Therefore, the degradation of xanthan gum can result in improved cleaning processes, such as the easier removal of stains containing gums, such as xanthan gum. Thus, the detergent composition comprising endoglucanases and/or xanthan lyases, as described herein, can be used to degrade xanthan gum. Degradation of xanthan gum can preferably be measured using the viscosity reduction assay (e.g. ViPr assay) or alternatively as described in examples 2 and 3 herein.

Endoglucanase activity may alternatively be measured by assessment of reducing ends on xanthan gum pre-treated with xanthan lyase using the colorimetric assay developed by Lever (Anal. Biochem. 47: 273-279, 1972). A preferred embodiment is the use of 0.1% xanthan gum pre-treated with xanthan lyase. Degradation of xanthan gum pre-treated with xanthan lyase may be determined by calculating difference between blank and sample, wherein a difference of more than 0.5 mAU, preferably more than 0.6 mAU, more preferably more than 0.7 mAU or even more preferably more than 0.8 mAU, shows degradation of xanthan gum pre-treated with xanthan lyase.

Xanthan lyase activity may alternatively be measured by assessment of reducing ends liberated from xanthan gum using the colorimetric assay developed by Lever (Anal. Biochem. 47: 273-279, 1972). A preferred embodiment is the use of 0.1% xanthan gum. Degradation of xanthan gum may be determined by calculating difference between blank and sample wherein a difference of more than 0.1 mAU, preferably more than 0.15 mAU, more preferably more than 0.2 mAU or even more preferably more than 0.25 mAU shows degradation of xanthan gum.

Endoglucanase and xanthan lyase activity may alternatively be measured by assessment of reducing ends liberated from xanthan gum using the colorimetric assay developed by Lever (Anal. Biochem. 47: 273-279, 1972). A preferred embodiment is the use of 0.1% xanthan gum. Degradation of xanthan gum may be determined by calculating difference between blank and sample wherein a difference of more than 0.4 mAU, preferably more than 0.5 mAU, more preferably more than 0.6 mAU or even more preferably more than 0.8 mAU shows degradation of xanthan gum.

The disclosure also relates to methods for degrading xanthan gum comprising applying a detergent composition comprising one or more endoglucanase variants, as described herein, and/or one or more xanthan lyase variants, as described herein.

Use in Detergents

The present disclosure inter alia relates to the use of detergent compositions comprising endoglucanase variants and/or xanthan lyase variants, as described herein, in cleaning processes such as the laundering of textiles and fabrics (e.g. household laundry washing and industrial laundry washing), as well as household and industrial hard surface cleaning, such as dish wash. For this, the endoglucanase variants and/or xanthan lyase variants may be added to a detergent composition comprising of one or more detergent components.

The endoglucanase variants and/or xanthan lyase variants, as described herein, may be added to and thus become a component of a detergent composition. The detergent composition may be formulated, e.g., as a hand or machine laundry detergent composition for both household and industrial laundry cleaning, including a laundry additive composition suitable for pre-treatment of stained fabrics and a rinse added fabric softener composition, or be formulated as a detergent composition for use in general household or industrial hard surface cleaning operations, or be formulated for hand or machine (both household and industrial) dishwashing operations. In a specific aspect, the present disclosure relates to a detergent additive comprising endoglucanase variants and/or xanthan lyase variants, as described herein.

The disclosure also relates to methods for degrading xanthan gum on the surface of a textile or hard surface, such as dish wash, comprising applying a detergent composition, as described herein, to xanthan gum.

It has been contemplated that the use of an endoglucanase variant and/or xanthan lyase variant, as described herein, alone gives an enzyme detergency benefit, preferably an enzyme detergency benefit on xanthan gum.

In some aspects, the disclosure relates to the use of a detergent composition comprising one or more detergent components and an endoglucanase variant, as described herein, together with a xanthan lyase variant, as described herein.

Use of Enzyme Stabilizing Component

In a particular aspect, the present disclosure relates to the use of an enzyme stabilizing component for improving stability of an endoglucanase variant, as described herein, and/or a xanthan lyase variant, as described herein, and/or a mixture thereof in a detergent composition, in particular in a liquid laundry detergent composition, and wherein the enzyme stabilizing component is selected from the group consisting of polyols such as glycerol or 1,2-propylene glycol, alditols such as non-cyclic polyols having the formula HOCH₂[CH(OH)]ₙCH₂OH, mannitol, isomalt, lactit, sorbitol, xylitol, threitol, erythritol or arabitol, alkanol amines such as ethanol amine, or formates such as sodium formate, potassium formate, calcium formate or magnesium formate, preferably said enzyme stabilizing component is selected from glycerol and sodium formate, more preferably being sodium formate.

In a preferred embodiment, the present disclosure relates to the use of glycerol and/or sodium formate for improving stability of an endoglucanase variant, as described herein, and/or a xanthan lyase variant, as described herein, and/or a mixture thereof in a detergent composition.

In a particular preferred embodiment, the present disclosure relates to the use of sodium formate for improving stability of an endoglucanase variant, as described herein, and/or a xanthan lyase variant, as described herein, and/or a mixture thereof in a detergent composition.

In a further preferred embodiment, the present disclosure relates to the use of sodium formate for improving stability of an endoglucanase variant, as described herein, and a xanthan lyase variant, as described herein, in a detergent composition.

In an even preferred embodiment, the present disclosure relates to the use of sodium formate for improving stability of an endoglucanase variant and/or a xanthan lyase variant in a detergent composition, as described herein,

wherein the endoglucanase has at least 60% and less than 100% sequence identity to SEQ ID NO:1 and having a set of alteration selected from the group consisting of A559N+Y579W+T697G, K512P+A559N+Y579W+T697G, N18G+A71E+A186P+E408D+Y579W+I602T+A651P+A688G+V756Y, N18G+N189K+E408D+A559N+Y579W+A688G+T697G+V756Y+K921R+Y934G, S313D+E408D, R880K+N905D+K921R+Y934G, I302D+S313D+E408D+Y579W+I602T+A651P+T697G+R880K+K921R+Y934G, and N216Q+S313D+E408D+D476R+Y579W+I602T+F638N+A651P+T697G+W719R+R880K+T887K+K921R+Y934G, wherein said positions correspond to amino acid positions of SEQ ID NO:1;

wherein the xanthan lyase variant has at least 60% and less than 100% sequence identity to SEQ ID NO:2 and having a set of alteration selected from the group consisting of E229N+N672D+P752K+G753E+A769D+L775A+D801G+K875T+N892Y, E229S+N672D+G753E+S754E+A769D+L775A+D801G+K875T+N892Y, E229S+N672D+P752R+G753E+S754E+A769D+L775A+D801G+K875T+N892Y, A190Q+E229S+I234V+A624E+N672D+G753E+S754E+A769D+L775A+D801G+K875T, A190Q+E229S+T631N+N672D+I703L+P752K+G753E+A769D+L775A+D801G+K875T, A190Q+E229S+I234V+S582K+N672D+G753E+S754E+S757D+A769D+L775A+D801G+K875T+N892Y, E229S+N440K+S582K+A624E+S635E+N672D+G738L+G753E+S754E+S757D+A769D+L775A+D801G+K875T+N892Y, and S100D+E229S+K360G+D458S+S582K+N672D+G753E+S754E+S757D+A769D+L775A +D801G+A843P+K875T+N892Y+N1008D, wherein said positions correspond to amino acid positions of SEQ ID NO:2.

### EXAMPLES

### Example 1

### Construction, Expression and Purification of Endoglucanase Variants and Xanthan Lyase Variants

Endoglucanase variants, as described herein, and xanthan lyase variants, as described herein, have been constructed and produced as described in WO 2018/037064, WO 2018/037065 and WO 2019/162000 (all three applications are incorporated herein by reference).

### Example 2

### Detergent Stability Assay for Endoglucanase

GH9 endoglucanase (EG) activity (EC 3.2.1.4) was determined by reducing ends on xanthan gum pre-treated with xanthan lyase using the colorimetric assay developed by Lever (Anal. Biochem. 47: 273-279, 1972). Pre-treated xanthan gum is a modified form of the xanthan sugar, where the terminal pyruvated mannose from side chains is removed (prepared according to Nankai et al. (1999) from the source Keltran). The GH9 mature parent endoglucanase and its variants cleave at beta-(1,4)-glucosyl bonds in the glucan backbone of pretreated xanthan gum releasing glucans with a reducing end which can be determined by reaction with p-Hydroxybenzoic acid hydrazide (PAHBAH). The increase of color is proportional to the enzyme activity. Enzyme sample was mixed with detergent (as exemplified in Table 1) and stored at 30° C. for 8 weeks. After incubation, the enzyme-detergent sample was 100-fold diluted in assay buffer (50 mM MOPS, 4 mM CaCl₂, 0.01% Triton X-100, pH 7.0).

To assess the enzymatic activity, 50 μl of diluted enzyme-detergent sample was mixed with 50 μl 4 mg/ml modified xanthan gum. The samples had then been incubated at 50° C. for 1 hour. Finally, the level of reducing ends is estimated by adding 75 μl PAHBAH solution (15 mg/ml PAHBAH, 50 g/L potassium sodium tartrate tetrahydrate, 20 g/L NaOH), and the samples had then been incubated at 95° C. for 10 minutes. After cooling down to room temperature, the absorbance at 405 nm was measured.

TABLE 1
Liquid laundry detergent matrix

| | % Active | % Active |
|---|---|---|
| | matter raw | matter in |
| | material | formula |
| Water demin. | 100 | 2-8 |
| 1,2-Propandiol | 100 | 3-7 |
| Glycerol | 99.5 | 7-11 |
| Monoethanolamin | | 4-8 |
| LAS (anionic surfactant) | 96 | 12-28 |
| FAEO (nonionic surfactant) | 100 | 12-28 |
| Alkanolamine alkoxylated | 100 | 3-7 |
| palm kernel oil fatty acid | 100 | 3-8 |
| Polyethyleneimine | 80 | 2-6 |
| CP propylene glycol terephthalate liquid | 100 | 1-4 |
| sodium disulfite | 98.6 | 0.01-0.3 |
| DTPMP-xNa7 | 40 | 0.1-1.0 |
| Optical brightener | 90 | 0.1-1 |
| Perfume | 100 | 1-5 |
| Dye | t.q. | 0.001-0.005 |
| Without enzymes; Dosage: 1 g/L; pH = 8 | | |

### Example 3

### Detergent Stability Assay for Xanthan Lyase

Enzyme sample was mixed with detergent (as exemplified in Table 1) and stored at 30° C. for 8 weeks. After incubation, the enzyme-detergent sample was 50-fold diluted in dilution buffer (50 mM MOPS, 1 mM CaCl₂, pH 7.5).

To measure the enzyme activity of diluted enzyme-detergent samples, 50 μl of diluted samples were mixed with 50 μl of freshly prepared substrate solution (0.4% w/v xanthan gum was freshly prepared by dissolving 400 mg of xanthan gum in 100 ml of Milli Q water) and incubated at 40° C. for 1 hour.

After incubation, 75 μl of PAHBAH reagent (1.5% PAHBAH reagent was freshly prepared by dissolving 1.5 g of p-hydroxy benzoic acid hydrazide in a stock solution mix (stock solution mix containing 1.0 M Na₂CO₃, 0.17 M potassium sodium tartrate and 5 mM (Bi(NO3)3 x 5H20) was prepared by dissolving 106.99 g of Na₂CO₃, 47.98 g of potassium sodium tartrate and 2.42 mg of (Bi(NO₃)₃×5H₂O) in Milli Q water for a final volume of 1000 ml)) was added, and the sample was incubated at 90° C. for 10 minutes, followed by subsequent cooling at 10° C. Afterwards, absorbance was measured at 405 nm (Infinite M1000 reader; TECAN, Switzerland).

### Example 4

### Residual Activity

The in-detergent stability was determined by measuring the enzymatic activity present in detergent fresh, after the enzymes have been added to the detergent and also after storage of the enzymes containing detergent at 30° C.

The residual activity (RA) was calculated as the percentage of enzymatic activity remaining after incubation at 30° C. relative to the enzymatic activity after freshly formulating the enzyme.

TABLE 2
Residual activity

| | Enzyme | Xanthan lyase | Endoglucanase |
|---|---|---|---|
| | stabilizing | activity after | activity after |
| | component | 8 weeks [%] | 8 weeks [%] |
| Detergent matrix S1 | None | 43% | 15% |
| Detergent matrix S2 | 5 wt.-% glycerol | 73% | 20% |
| Detergent matrix S3 | 1 wt.-% sodium | 76% | 61% |
| | formate | | |

S1: Detergent matrix as shown in Table 1 with 0.7 wt.-% (t.q.) of enzyme mix containing endoglucanase and xanthan lyase, as described herein; pH=8

S2: Detergent matrix as shown in Table 1 with additional 5 wt.-% glycerol and 0.7 wt.-% (t.q.) of enzyme mix containing endoglucanase and xanthan lyase; pH =8

S3: Detergent matrix as shown in Table 1 with 1 wt.-% sodium formate and 0.7 wt.-% (t.q.) of enzyme mix containing endoglucanase and xanthan lyase; pH =8

All three samples, S1, S2 and S3, had been stored at 30° C. for 8 weeks. After storage, residual activity of endoglucanase and xanthan lyase had been determined as described above.

From Table 2, it can be seen that endoglucanase as well as xanthan lyase can be stabilized by rather low sodium formate concentrations, whereas elevated glycerol concentration only has a stabilizing effect on xanthan lyase.

Using as low concentration of enzyme stabilizer as possible is especially advantageous in compacted dosage forms, like in particular unit dose, pouches and caps. In this respect, sodium formate shows surprisingly good stabilizing results while being applied in rather low concentration.

1. A detergent composition comprising:
(A) an endoglucanase variant comprising an alteration at one or more positions in one or more regions selected from the group consisting of:
region 7 corresponding to amino acids 612 to 660 of SEQ ID NO:1,
region 1 corresponding to amino acids 95 to 105 of SEQ ID NO:1,
region 2 corresponding to amino acids 115 to 138 of SEQ ID NO:1,
region 3 corresponding to amino acids 210 to 251 of SEQ ID NO:1,
region 4 corresponding to amino acids 267 to 301 of SEQ ID NO:1,
region 5 corresponding to amino acids 339 to 361 of SEQ ID NO:1,
region 6 corresponding to amino acids 547 to 595 of SEQ ID NO:1,
region 8 corresponding to amino acids 806 to 828 of SEQ ID NO:1,
region 9 corresponding to amino acids 839 to 1042 of SEQ ID NO:1,
region 10 corresponding to amino acids 1 to 94 of SEQ ID NO:1,
region 11 corresponding to amino acids 106 to 114 of SEQ ID NO:1,
region 12 corresponding to amino acids 139 to 209 of SEQ ID NO:1,
region 13 corresponding to amino acids 252 to 266 of SEQ ID NO:1,
region 14 corresponding to amino acids 302 to 338 of SEQ ID NO:1,
region 15 corresponding to amino acids 362 to 546 of SEQ ID NO:1,
region 16 corresponding to amino acids 596 to 611 of SEQ ID NO:1,
region 17 corresponding to amino acids 661 to 805 of SEQ ID NO:1,
region 18 corresponding to amino acids 829 to 838 of SEQ ID NO:1, and
region 19 corresponding to amino acids 1043 to 1055 of SEQ ID NO:1;
and/or
(B) a xanthan lyase variant comprising an alteration at one or more positions in one or more regions selected from the group consisting of:
region 3 corresponding to amino acids 731 to 803 of SEQ ID NO:2,
region 1 corresponding to amino acids 154 to 176 of SEQ ID NO:2,
region 2 corresponding to amino acids 614 to 658 of SEQ ID NO:2,
region 4 corresponding to amino acids 807 to 846 of SEQ ID NO:2,
region 5 corresponding to amino acids 872 to 885 of SEQ ID NO:2,
region 6 corresponding to amino acids 903 to 1004 of SEQ ID NO:2,
region 7 corresponding to amino acids 1 to 153 of SEQ ID NO:2,
region 8 corresponding to amino acids 177 to 613 of SEQ ID NO:2,
region 9 corresponding to amino acids 659 to 730 of SEQ ID NO:2,
region 10 corresponding to amino acids 804 to 806 of SEQ ID NO:2,
region 11 corresponding to amino acids 847 to 871 of SEQ ID NO:2,
region 12 corresponding to amino acids 886 to 902 of SEQ ID NO:2, and
region 13 corresponding to amino acids 1005 to 1037 of SEQ ID NO:2; and
(C) an enzyme stabilizing component;
wherein the endoglucanase variant (A) has at least 60% and less than 100% sequence identity to SEQ ID NO:1;
wherein the xanthan lyase variant (B) has at least 60% and less than 100% sequence identity to SEQ ID NO:2;
wherein the enzyme stabilizing component (C) comprises a formate.

2. The detergent composition according to claim 1, wherein said endoglucanase variant has at least 75% and less than 100% sequence identity to SEQ ID NO:1.

3. The detergent composition according to claim 1, wherein said endoglucanase variant comprises an alteration at one or more positions selected from the group consisting of 4, 17, 18, 20, 51, 53, 55, 56, 60, 63, 71, 79, 87, 92, 99, 120, 125, 126, 130, 137, 182, 186, 189, 192, 213, 216, 221, 226, 228, 230, 231, 232, 233, 235, 240, 243, 247, 249, 278, 279, 281, 283, 285, 289, 292, 294, 298, 302, 311, 313, 333, 346, 353, 358, 386, 387, 388, 390, 403, 408, 410, 416, 441, 448, 451, 471, 472, 476, 489, 507, 512, 515, 538, 555, 556, 557, 558, 559, 560, 561, 562, 563, 564, 567, 568, 570, 575, 578, 579, 580, 581, 583, 589, 590, 591, 592, 593, 595, 598, 599, 602, 603, 605, 607, 609, 616, 627, 630, 631, 635, 636, 638, 639, 640, 641, 642, 643, 644, 651, 676, 683, 688, 690, 694, 698, 699, 706, 711, 713, 719, 720, 744, 749, 754, 756, 760, 781, 786, 797, 810, 811, 812, 815, 823, 824, 825, 827, 828, 833, 834, 835, 837, 843, 848, 868, 869, 870, 871, 872, 873, 874, 880, 881, 883, 884, 885, 887, 888, 890, 892, 894, 898, 905, 906, 912, 920, 921, 924, 926, 927, 928, 932, 933, 934, 935, 937, 938, 939, 940, 941, 942, 943, 946, 948, 950, 952, 953, 954, 956, 957, 960, 966, 971, 972, 980, 989, 991, 994, 995, 998, 999, 1006, 1009, 1010, 1011, 1029, 1030, 1031, 1032, 1035, 1037, 1038, 1040, 1041, 1042, 1044, 1045,. and 1048, wherein said positions correspond to amino acid positions of SEQ ID NO:1.

4. The detergent composition according to claim 1, wherein said endoglucanase variant comprises one or more amino acid substitutions selected from the group consisting of V4T, S17A, N18G, F20P, F20N, F20G, F20Y, K51Q, K51H, E53Y, E53P, E53G, Y55M, Y55D, V56M, Y60F, S63F, A71E, T87R, T92S, K99R, A120P, I125V, A126R, S128X, N129D, K130R, F137L, H182Y, A186P, N189K, K192N, K213R, N216D, N216Q, N216R, A221R, L226K, K228E, K228I, G230F, G230L, G230A, G230H, G230N, G230W, G230T, F231Y, F231N, V232R, V232G, L233H, H235D, N240Q, G243K, G243R, D247N, A249N, A278S, G279E, K281F, K281V, K281Y, K281H, K281Q, K281N, K281W, K281T, K281R, A283D, N285G, N285L, N285M, N285S, N285P, N285T, N285Y, N285H, N285K, N285D, N285W, N285R, Q289E, T292A, T292F, T292L, T292I, T292V, T292S, T292P, T292Y, T292Q, T292N, T292K, T292D, T292G, A294V, F297L, Q298E, I302D, I302H, I302V, I302M, H311N, S313D, W333L, A346H, A346D, T353D, A386P, I387T, K388R, K390Q, I403Y, E408D, E408N, E408S, E408P, E408A, E408G, P410G, Q416S, Q416D, N441G, A448E, A448W, A448S, K451Q, K451S, G471S, S472Y, D476R, Q489P, K507R, K512P, S515V, S538C, L555Q, G556S, G557R, N558P, N558F, N558K, N558D, N558M, N558Q, N558I, N558Y, N558H, N558E, A559N, A559F, A559M, A559P, A559Y, A559H, A559Q, A559D, A559R, A559G, A559I, A559S, A559K, S560F, S560P, S560K, S560G, S560D, T561E, T561Q, T561S, T561D, T561P, G562W, G562P, G563E, A564I, A564Y, A564H, A564Q, A564K, A564E, A564D, E565M, V567F, V567P, K568R, L569F, L569Y, L569D, L569E, P570F, P570L, P570I, P570M, P570V, P570S, P570T, P570A, P570Y, P570H, P570Q, P570N, P570K, P570E, P570W, P570R, P570G, I575V, I575M, I575A, I575D, I575E, I576F, I576M, I576P, D578R, Y579F, Y579W, V580L, T581M, D583M, Q589G, P590S, P590T, P590E, E591L, G592D, S593P, S593H, S593Q, S593N, S593K, S593D, S593E, S593R, S595L, S598Q, A599S, I602T, I602D, V603P, S605T, S607C, G609E, S616G, S616D, K627L, K627M, K627V, K627S, K627T, K627Q, K627R, I630F, I630V, 1630Y, K631R, K631A, T633V, D635L, D635M, D635T, D635A, D635P, D635N, D635K, D635E, D635G, D635W, S636L, S636M, S636A, S636H, S636Q, S636N, S636K, S636R, F638N, F638Y, T639D, F638I, F638V, F638T, F638L, F638H, F638M, T639V, T639S, T639L, T639I, T639M, T639A, T639E, T639W, T639G, T639Y, T639P, T640S, Y641E, S642T, S642N, N643D, N643H, N643T, T644F, A651P, A651S, D676H, Q683E, A688G, Y690F, T694A, T697G, R698W, T699A, T706Q, T711S, T711V, T711Y, K713R, W719R, K720H, K744H, K744Q, A749T, K754R, V756Y, V756H, S760G, T781M, N786K, T797S, S810Q, S810R, A811S, V812F, V812I, V812M, V812W, V812R, N815V, N815Y, N815E, N815W, N815R, S823Q, A824T, A824D, T825G, T825N, T825W, T825A, T825D, V827I, V827M, V827S, N828D, N833D, Q834E, S835A, S835D, V837I, T843V, N848D, A868E, A869V, D870F, D870L, D870I, D870M, D870V, D870S, D870T, D870Y, D870H, D870Q, D870N, D870K, D870E, D870W, D870R, D870G, P871F, P871L, P871I, P871M, P871V, P871S, P871T, P871A, P871Y, P871H, P871Q, T872S, T872F, T872A, T872Y, T872H, T872Q, T872N, T872K, T872D, T872E, T872W, T872R, T872G, D873K, D873E, T874V, T874S, T874P, T874A, T874H, T874Q, T874N, T874K, R880K, V881Q, V881T, T883R, T883V, T883C, T883K, Y884H, A885F, A885Q, A885N, T887L, T887I, T887S, T887H, T887R, T887K, L888M, V890R, T892P, T892V, K894E, R898Q, N905D, F906A, Q912V, N920P, N920D, K921R, K921E, K921D, A924D, V926F, V926P, K927R, S928D, T932A, N933V, N933S, Y934G, Y934M, Y934S, Y934A, Y934Q, Y934N, Y934E, Y934W, Y934R, T935W, A937F, A937V, A937S, A937T, A937Q, A937D, A937E, V938I, K939I, K939V, D940E, N941S, N941H, N941D, A942P, A942E, D943Y, D943H, G946R, K948R, K948E, R950V, R950H, R950N, F952S, F952W, N953Y, G954L, Q956Y, Q956S, A957L, A957P, Y960F, A964N, A964C, N966P, N966C, G971A, T972K, Q974K, Q974C, M980I, Q989I, Q991L, Q991I, Q991M, Q991V, Q991T, Q991K, Q991C, G994D, G994N, S995I, S995V, S995Q, S995R, S995C, G998V, G998A, T999R, S1006T, S1006A, S1006K, S1006R, K1009E, Y1010W, L1011M, L1011S, L1011A, L1011Q, L1011N, L1011D, L1011E, R1029N, F1030M, K1031I, K1031S, K1031T, K1031H, V1032G, K1035A, A1037E, A1037W, S1038L, S1038I, S1038G, L1040N, L1040E, G1041F, G1041R, Y1042N, L1044F, L1044S, L1044N, L1044W, P1045Q, P1045W, and F1048W, preferably selected from the group consisting of S17A, F20P, N216D, N216Q, A283D, I302D, H311N, S313D, E408D, D476R, Y579W, I602T, K627R, S636N, S636K F638I, F638N, A651P, A688G, T697G, W719R, V756Y, R880K, V881Q, T883R, T887K, F906A, K921R, Y934G, A937E, and Q956Y, wherein said positions correspond to amino acid positions of SEQ ID NO:1.

5. The detergent composition according to claim 1, wherein said endoglucanase variant comprises a set of alterations selected from the group consisting of:
A559K+N558K+S560F+T561P+G562W,
A559N+P570Q,
A559N+P570T,
A559N+Y579F,
A559N+Y579F+K627R, A559N+Y579W, A559N+Y579W+K99R,
A559N+Y579W+S128X+K627R,
A559N+Y579W+S128X+S636N,
A559N+Y579W+K281R,
A559N+Y579W+Q416D+S636N, A559N+Y579W+S560P,
A559N+Y579W+A564I, A559N+Y579W+P570N,
A559N+Y579W+P570K,
A559N+Y579W+P570R,
A559N+Y579W+P570A,
A559N+Y579W+P570T,
A559N+Y579W+P570S,
A559N+Y579W+P570Q,
A559N+Y579W+P570H,
A559N+Y579W+S616D,
A559N+Y579W+S616D+K627R,
A559N+Y579W+S616D+S636K,
A559N+Y579W+S616D+A651P,
A559N+Y579W+S616D+K921R,
A559N+Y579W+S616D+K921R+Y934G, A559N+Y579W+K627R,
A559N+Y579W+K627R+S636N+A651P,
A559N+Y579W+K627R+S636N+K921R,
A559N+Y579W+K627R+K921R,
A559N+Y579W+K627M,
A559N+Y579W+K627M+A651P,
A559N+Y579W+S636N,
A559N+Y579W+S636N+A651P,
A559N+Y579W+S636N+K921R,
A559N+Y579W+S636K+A651P,
A559N+Y579W+S636K+K921R,
A559N+Y579W+S636L,
A559N+Y579W+F638I,
A559N+Y579W+A651P,
A559N+Y579W+A651P+K921R+Y934G,
A559N+Y579W+A651P+Y934G,
A559N+Y579W+D870M,
A559N+Y579W+K921R,
A559N+Y579W+K921R+A651P,
A559N+Y579W+K921R+Y934G,
A559N+Y579W+Y934G,
A559N+Y579W+K948E,
A559N+Y579W+K1009E,
A559N+K627R,
Y579F+A564E,
Y579W+K99R,
Y579W+G562P,
Y579W+A564D,
Y579W+P570K,
Y579W+P570Q,
Y579W+K627R,
Y579W+S636N,
Y579W+F638I,
Y579W+A651P,
Y579W+K921R,
Y579W+Y934G,
Y579W+K948E,
Y579W+K1009E,
K627R+K51Q,
K627R+K451S,
K627R+P570Q,
K627R+P570T,
K627R+S636N,
K627R+F638I,
K627R+A885F,
K627R+Y934G,
S636N+P570T,
S636N+K921R,
S636N+Y934G,
F638I+P570K,
F638I+P570Q,
F638I+P570T,
F638I+A651P,
F638I+Y934G,
F638I+K921R,
A651P+P570Q,
A651P+P570T,
A885F+P570T,
A885F+Y934G,
K921R+P570Q,
K921R+P570T,
Y934G+P570T, wherein said positions correspond to amino acid positions of SEQ ID NO:1.

6. The detergent composition according to claim 1, wherein said endoglucanase variant has activity on xanthan gum pre-treated with xanthan lyase.

7. The detergent composition according to claim 1, wherein said xanthan lyase variant has at least 75% and less than 100% sequence identity to SEQ ID NO:2.

8. The detergent composition according to claim 1, wherein said xanthan lyase variant comprises an alteration at one or more positions selected from the group consisting of 9, 15, 18, 46, 58, 66, 89, 95, 100, 106, 109, 155, 159, 183, 188, 190, 203, 204, 221, 229, 234, 238, 240, 242, 243, 257, 258, 284, 291, 293, 316, 317, 320, 324, 329, 333, 339, 341, 352, 354, 360, 372, 377, 399, 400, 419, 440, 450, 451, 454, 458, 481, 492, 505, 533, 567, 568, 576, 578, 579, 582, 620, 624, 626, 631, 635, 649, 650, 656, 664, 672, 703, 722, 726, 727, 728, 738, 745, 752, 753, 754, 757, 769, 775, 777, 779, 782, 786, 789, 792, 796, 799, 801, 819, 824, 843, 851, 855, 856, 867, 875, 887, 892, 899, 900, 901, 902, 903, 911, 912, 915, 919, 921, 923, 925, 927, 928, 930, 933, 941, 966, 991, 1008, and 1016, wherein said positions correspond to amino acid positions of SEQ ID NO:2.

9. The detergent composition according to claim 1, wherein said xanthan lyase variant comprises one or more amino acid substitutions selected from the group consisting of K9R, N15T, T18D, L46D, A58L, S66H, Q89Y, K95E, S100D, N106Y, Q109R, Q109D, Q109F, Q109K, Q109A, Y155E, A159P, K183Q, K183R, V188I, A190Q, A203P, K204R, A221P, E229N, E229S, E229V, I234V, I238W, I238L, I238M, 1240W, N242S, G243V, Y257W, R258E, R284G, K291R, A293G, A293P, K316R, R317K, K320R, L324Q, K329R, K333R, L339M, I341P, V352I, S354P, K360G, K360R, Q372H, F377Y, N399K, K400R, F419Y, N440K, D450P, K451E, K451R, A454V, D458S, K481R, A492H, A492L, T505I, L533I, K567R, G568A, S578K, S578N, S578R, S579R, S579K, S582K, K620R, A624E, A626Q, T631N, S635E, S635T, T649V, T649K, Q650G, I656V, T664K, N672D, I703L, I722F, P726Q, T727P, M728V, G738L, K745R, P752R, P752K, G753E, G753Q, G753S, S754E, S754L, S754Q, S754R, S757D, S757P, S757E, A769D, A769T, A769R, L775A, L775F, L775I, L775M, L775Q, L775S, L775Y, D777R, P779V, Y782I, N786K, G789R, K792W, K792Y, K792V, K792A, N796Q, A799H, D801G, K819R, K824R, A843P, S851F, K855R, E856D, P867S, K875T, K875E, K887R, N892Y, N892W, N892F, G899S, I900G, D901A, T902F, T903A, T903Q, A911M, A911V, A911S, A912T, A912I, A912Y, T915S, T915V, T915Q, T919D, T919F, T919G, T921R, T921S, T923D, T923H, T925D, T925Q, T925R, T927K, D928W, Y930F, Y930H, Y930L, D933M, G941D, G941E, A966P, N991D, V998K, N1008D, and K1016T, wherein said positions correspond to amino acid positions of SEQ ID NO:2.

10. The detergent composition according to claim 1, wherein said xanthan lyase variant comprises an alteration at one or more positions selected from the group consisting of 624, 631, 635, 649, 656, 738, 752, 753, 754, 757, 769, 775, 777, 800, 801, 843, 875, 911 and 915, and/or an alteration at one or more positions selected from the group consisting of 89, 100, 190, 229, 234, 352, 360, 399, 440, 458, 492, 567, 582, 664, 672, 703, 728, 892, 1008, and 1016, wherein said positions correspond to amino acid positions of SEQ ID NO:2.

11. The detergent composition according to claim 1, wherein said xanthan lyase variant comprises one or more substitutions selected from the group consisting of Q89Y, S100D, A190Q, E229S, I234V, V352I, K360G, N399K, N440K, D458S, A492H, A492L, K567R, S582K, T664K, N672D, I703L, M728V, N892Y, N1008D, and K1016T, and/or one or more substitutions selected from the group consisting of A624E, T631N, S635E, T649K, I656V, G738L, P752K, P752R, G753E, S754E, S754R, S757D, A769D, L775A, D777R, V800P, D801G, A843P, K875T, A911V, and T915A, wherein said positions correspond to amino acid positions of SEQ ID NO:2.

12. The detergent composition according to claim 1, wherein said xanthan lyase variant comprises a set of alterations selected from the group consisting of:
E229N+N672D+P752K+G753E+A769D+L775A+D801G+K875T+N892Y, E229S+N672D+G753E+S754E+A769D+L775A+D801G+K875T+N892Y, E229S+N672D+P752R+G753E+S754E+A769D+L775A+D801 G+K875T+N892Y,
A190Q+E229S+I234V+A624E+N672D+G753E+S754E+A769D+L775A+D801 G+K875T,
A190Q+E229S+T631N+N672D+I703L+P752K+G753E+A769D+L775A+D801 G+K875T,
A190Q+E229S+I234V+S582K+N672D+G753E+S754E+S757D+A769D+L775A+D801G+K875T+N892Y,
E229S+N440K+S582K+A624E+S635E+N672D+G738L+G753E+S754E+S757D+A769D+L775A+D801G+K875T+N892Y, and
S100D+E229S+K360G+D458S+S582K+N672D+G753E+S754E+S757D+A769D+L775A+D801G+A843P+K875T+N892Y+N1008D, wherein said positions correspond to amino acid positions of SEQ ID NO:2.

13. The detergent composition according to claim 1, wherein the formate is selected from sodium formate, potassium formate, calcium formate, magnesium formate, or combinations thereof.

14. The detergent composition according to claim 1, wherein:
(A) the endoglucanase variant has at least 75% and less than 100% sequence identity to SEQ ID NO:1 and having a set of alteration selected from the group consisting of:
(A1) A559N+Y579W+T697G,
(A2) K512P+A559N+Y579W+T697G,
(A3) N18G+A71E+A186P+E408D+Y579W+I602T+A651P+A688G+V756Y,
(A4) N18G+N189K+E408D+A559N+Y579W+A688G+T697G+V756Y+K921R+Y934G,
(A5) S313D+E408D,
(A6) R880K+N905D+K921R+Y934G,
(A7) I302D+S313D+E408D+Y579W+I602T+A651P+T697G+R880K+K921R+Y934G, and
(A8) N216Q+S313D+E408D+D476R+Y579W+I602T+F638N+A651P+T697G+W719R+R880K+T887K+K921R+Y934G,
wherein said positions correspond to amino acid positions of SEQ ID NO:1;
(B) the xanthan lyase variant has at least 75% and less than 100% sequence identity to SEQ ID NO:2 and having a set of alteration selected from the group consisting of:
(B1) E229N+N672D+P752K+G753E+A769D+L775A+D801G+K875T+N892Y,
(B2) E229S+N672D+G753E+S754E+A769D+L775A+D801G+K875T+N892Y,
(B3) E229S+N672D+P752R+G753E+S754E+A769D+L775A+D801G+K875T+N892Y,
(B4) A190Q+E229S+I234V+A624E+N672D+G753E+S754E+A769D+L775A+D801G+K875T,
(B5) A190Q+E229S+T631N+N672D+I703L+P752K+G753E+A769D+L775A+D801G+K875T,
(B6) A190Q+E229S+I234V+S582K+N672D+G753E+S754E+S757D+A769D+L775A+D801G+K875T+N892Y,
(B7) E229S+N440K+S582K+A624E+S635E+N672D+G738L+G753E+S754E+S757D+A769D+L775A+D801G+K875T+N892Y, and
(B8) S100D+E229S+K360G+D458S+S582K+N672D+G753E+S754E+S757D+A769D+L775A+D801G+A843P+K875T+N892Y+N1008D,
wherein said positions correspond to amino acid positions of SEQ ID NO:2; and
(C) the enzyme stabilizing component is sodium formate.

15. The detergent composition according to claim 14, wherein the endoglucanase variant and/or the xanthan lyase variant do not comprise any further substitution.

16. The detergent composition according to claim 1, wherein said endoglucanase variant and/or said xanthan lyase variant has improved stability in the presence of said enzyme stabilizing component.

17. The detergent composition according to claim 1, wherein said detergent compositions comprises 0.000001 to 10 wt.-% of said endoglucanase variant and/or 00.000001 to 10 wt. % of said xanthan lyase variant and/or 0.001 to 20 wt.-% of said enzyme stabilizing agent; and/or wherein said detergent composition comprises said endoglucanase variant and/or said xanthan lyase variant and said enzyme stabilizing agent in a weight ratio (enzyme: stabilizer) of 0.00001:1 to 0.1:2.

18. The detergent composition according to claim 1, wherein the detergent composition is an automatic dishwashing composition, hand dishwashing composition, or laundry detergent composition.

19. The detergent composition according to claim 1, wherein said detergent composition is in unit dose.

20. (canceled)

21. (canceled)

22. (canceled)

23. (canceled)

24. (canceled)
